(19) United States
(12) Patent Application Publication
(10) Pub. No.: US 2025/0145984 A1
(43) Pub. Date: May 8, 2025

(54) METHODS OF REDUCING LATERAL DIFFUSION OF AN ANALYTE

(71) Applicant: 10x Genomics, Inc., Pleasanton, CA (US)

(72) Inventors: Zixue Ma, Pleasanton, CA (US); Augusto Manuel Tentori, Dublin, CA (US)

(21) Appl. No.: 18/939,142

(22) Filed: Nov. 6, 2024

Related U.S. Application Data
(60) Provisional application No. 63/596,449, filed on Nov. 6, 2023.

(51) Int. Cl. C12N 15/10

(52) U.S. Cl. CPC C12N 15/1065

(57) ABSTRACT

Provided herein are methods using an anisotropic structure within or in proximity to a biological sample. Such methods can include identifying a location of an analyte in a biological sample or reducing mislocalization of an analyte in a biological sample.

### CROSS-REFERENCE TO RELATED APPLICATIONS

This application claims the benefit of U.S. Provisional Application No. 63/596,449 filed Nov. 6, 2023, which is herein incorporated by reference in its entirety.

### SEQUENCE LISTING

This application contains a Sequence Listing that has been submitted electronically as an XML file named “47706-0370001_ST26_SL.XML.” The XML file, created on Nov. 6, 2024, is 7,039 bytes in size. The material in the XML file is hereby incorporated by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

The present disclosure provides methods for mitigating transcript mislocalization (TML) from a biological sample, such as a tissue or cell, by introducing an anisotropic structure to a substrate and/or biological sample, thereby decreasing TML and/or resulting in higher resolution analyte expression data from the biological sample.

### SUMMARY

Spatial transcriptomics (ST) can be used to reduce a three-dimensional (3D) distribution of molecules within a sample into a two-dimensional (2D) image. To do so, a 2D image can be obtained by capturing target molecules that migrate vertically from their original location (e.g., such as within the sample) onto a spatially tagged array. Migration of molecules is typically a passive event, and analytes can migrate in non-vertical directions thereby resulting in a reduction of resolution, accuracy, and/or sensitivity. Mitigating such transcript mislocalization (e.g., diffusion, flow, convection based) would have an impact on the quality of spatial data. Transcript mislocalization (TML) can result in incorrect registration of spatial information which can lead to incorrect detection of gene expression and potentially cell type identification in spatial data.

Described herein are methods that can reduce mislocalization of an analyte in a biological sample. Such methods can provide improved binding or capture of the target analyte.

Thus, included herein are methods for determining the location of an analyte in a biological sample that include: (a) providing the biological sample on a substrate, wherein the substrate comprises a plurality of capture probes, and wherein a hydrogel comprising a plurality of anisotropic structures is disposed between the biological sample and a surface of the substrate comprising the plurality of capture probes; (b) hybridizing or contacting the analyte, or a proxy thereof, with at least one capture probe of the plurality of capture probes, wherein the at least one capture probe comprises a capture domain and a spatial barcode; and (c) determining (i) the spatial barcode or a complement thereof, and (ii) all or a portion of the analyte from the biological sample, and using the determined sequences of (i) and (ii) to determine the location of the analyte in the biological sample.

In some embodiments, the providing step (a) comprises (i) providing an initial substrate comprising the plurality of capture probes and the hydrogel comprising the plurality of anisotropic structures; and (ii) contacting the biological sample with the hydrogel, thereby providing the biological sample on the substrate. In some embodiments, the hydrogel comprising the plurality of anisotropic structures is prepared by (i) applying an external field to a pre-gel solution comprising a plurality of gelators, optionally wherein the pre-gel solution is disposed on a surface of the initial substrate, and (ii) optionally applying a reagent to promote gelation of the pre-gel solution, thereby providing the hydrogel comprising the plurality of anisotropic structures.

In some embodiments, the providing step (a) comprises (i) contacting the biological sample with a pre-gel solution that is disposed on a surface of an initial substrate comprising the plurality of capture probes, wherein the pre-gel solution comprises a plurality of gelators; (ii) applying an external field to the pre-gel solution, thereby providing the hydrogel comprising the plurality of anisotropic structures and providing the biological sample on the substrate.

In some embodiments, the providing step (a) comprises (i) contacting the biological sample with a pre-gel solution that is disposed on a surface of an initial substrate comprising the plurality of capture probes, wherein the pre-gel solution comprises a plurality of gelators; (ii) applying an external field to the pre-gel solution, thereby providing an anisotropic phase within the pre-gel solution; and (iii) applying a reagent to promote gelation of the pre-gel solution, thereby providing the hydrogel comprising the plurality of anisotropic structures and providing the biological sample on the substrate. In some embodiments, the reagent comprises a salt or an acidifier, optionally wherein the salt is selected from a Ca2+ or Mg2+ salt and/or optionally wherein the acidifier is selected from an acid or glucono-delta-lactone.

In some embodiments, the biological sample is permeabilized to allow the analyte, or the proxy thereof, in the biological sample to interact with the at least one capture probe of the plurality of capture probes; or wherein the method further comprises permeabilizing the biological sample to allow the analyte, or the proxy thereof, in the biological sample to interact with the at least one capture probe of the plurality of capture probes, wherein the permeabilizing is performed before or during step (b).

Also provided herein are methods for determining the location of an analyte in a biological sample that include: (a) providing the biological sample on a first substrate; (b) aligning the first substrate with a second substrate comprising an array and a hydrogel, such that at least a portion of the biological sample is aligned with at least a portion of the array, and such that at least a portion of the hydrogel is disposed between the biological sample and the second substrate, wherein the array comprises a plurality of capture probes, and wherein the hydrogel comprises a plurality of anisotropic structures; (c) when the biological sample is aligned with at least a portion of the array and at least a portion of the hydrogel, hybridizing or contacting the analyte, or a proxy thereof, with at least one capture probe of the plurality of capture probes, wherein the at least one capture probe comprises a capture domain and a spatial barcode; and (d) determining (i) the spatial barcode or a complement thereof, and (ii) all or a portion of the analyte from the biological sample, and using the determined sequences of (i) and (ii) to determine the location of the analyte in the biological sample.

In some embodiments, any one of the methods described herein further comprises permeabilizing the biological sample to allow the analyte, or the proxy thereof, in the biological sample to interact with at least one capture probe of the plurality of capture probes, wherein the permeabilizing is performed before or during step (c).

Also provided herein are methods for determining the location of an analyte in a biological sample that include: (a) providing the biological sample on a first substrate; (b) aligning the first substrate with a second substrate comprising an array, such that at least a portion of the biological sample is aligned with at least a portion of the array, and such that at least a portion of a pre-gel solution is disposed between the biological sample and the second substrate, wherein the array comprises a plurality of capture probes; (c) applying an external field to the pre-gel solution and optionally applying a reagent to promote gelation of the pre-gel solution, thereby providing a hydrogel comprising a plurality of anisotropic structures; (d) when the biological sample is aligned with at least a portion of the array and at least a portion of the hydrogel, hybridizing or contacting the analyte, or a proxy thereof, with at least one capture probe of the plurality of capture probes, wherein the at least one capture probe comprises a capture domain and a spatial barcode; and (c) determining (i) the spatial barcode or a complement thereof, and (ii) all or a portion of the analyte from the biological sample, and using the determined sequences of (i) and (ii) to determine the location of the analyte in the biological sample.

In some embodiments, any one of the methods provided herein further comprises permeabilizing the biological sample to allow the analyte, or the proxy thereof, in the biological sample to interact with at least one capture probe of the plurality of capture probes, wherein the permeabilizing is performed before or during step (d).

Also provided herein are methods for determining the location of a nucleic acid in a biological sample that include: (a) contacting the biological sample with a substrate, wherein the substrate comprises: (i) a plurality of capture probes and (ii) a hydrogel comprising plurality of anisotropic structures, such that the hydrogel is disposed between the biological sample and a surface of the substrate comprising the plurality of capture probes; (b) hybridizing or contacting the nucleic acid of the biological sample, or a proxy thereof, with at least one capture probe of the plurality of capture probes, wherein the at least one capture probe comprises a capture domain and a spatial barcode; (c) extending a 3′ end of the at least one capture probe using the nucleic acid of the biological sample, or the proxy thereof, bound to the capture domain as a template to generate an extended capture probe; and (d) determining (i) all or a part of a nucleotide sequence of the nucleic acid of the biological sample bound to the capture domain, or a complement thereof, and (ii) a nucleotide sequence of the spatial barcode, or a complement thereof, and using the determined nucleotide sequences of (i) and (ii) to determine the location of the nucleic acid in the biological sample.

In some embodiments, any one of the methods provided herein further comprises permeabilizing the biological sample to allow the nucleic acid in the biological sample, or the proxy thereof, to interact with the at least one capture probe of the plurality of capture probes, wherein the permeabilizing is performed before or during step (b). In some embodiments, any one of the methods provided herein further comprises generating a complement of the extended capture probe. In some embodiments, any one of the methods provided herein further comprises releasing the complement of the extended capture probe and optionally amplifying the complement of the extended capture probe.

Also provided herein are methods for determining the location of a nucleic acid in a biological sample that include: (a) contacting the biological sample with a substrate, wherein the substrate comprises a plurality of capture probes; (b) contacting the biological sample with a pre-gel solution that is disposed on a surface of the substrate; (c) applying an external field to the pre-gel solution and optionally applying a reagent to promote gelation of the pre-gel solution, thereby providing a hydrogel comprising a plurality of anisotropic structures; (d) hybridizing or contacting the nucleic acid of the biological sample, or a proxy thereof, with at least one capture probe of the plurality of capture probes, wherein the at least one capture probe comprises a capture domain and a spatial barcode; (c) extending a 3′ end of the at least one capture probe using the nucleic acid of the biological sample, or the proxy thereof, bound to the capture domain as a template to generate an extended capture probe; and (f) determining (i) all or a part of a nucleotide sequence of the nucleic acid of the biological sample bound to the capture domain, or a complement thereof, and (ii) a nucleotide sequence of the spatial barcode, or a complement thereof, and using the determined nucleotide sequences of (i) and (ii) to determine the location of the nucleic acid in the biological sample.

In some embodiments, any one of the methods provided herein further comprises permeabilizing the biological sample to allow the nucleic acid in the biological sample, or the proxy thereof, to interact with the at least one capture probe of the plurality of capture probes, wherein the permeabilizing is performed before or during step (d). In some embodiments, any one of the methods provided herein further comprises generating a complement of the extended capture probe. In some embodiments, any one of the methods provided herein further comprises releasing the complement of the extended capture probe and optionally amplifying the complement of the extended capture probe. In some embodiments, the nucleic acid is DNA. In some embodiments, the nucleic acid is RNA.

Also provided herein are methods for mitigating mislocalization of analytes captured on a spatial array that include: (a) providing the biological sample on a substrate, wherein the substrate comprises a spatial array comprising a plurality of capture probes, and wherein a hydrogel comprising a plurality of anisotropic structures is disposed between the biological sample and the spatial array; and (b) hybridizing or capturing an analyte, or the proxy thereof, using at least one capture probe of the plurality of capture probes, wherein the at least one capture probe comprises a capture domain and a spatial barcode, thereby mitigating mislocalization of the captured analyte, or the captured proxy thereof, and its subsequent mislocalization on the spatial array.

In some embodiments, step (b) comprises migration of the captured analyte, or the captured proxy thereof, wherein the migration comprises vertical migration between the biological sample and the spatial array. In some embodiments, any one of the methods provided herein further comprises: (c) determining (i) the spatial barcode or a complement thereof, and (ii) all or a portion of the sequence of the analyte from the biological sample.

In some embodiments, the plurality of anisotropic structures in the hydrogel comprises a polymeric network formed by assembling a plurality of gelators, thereby forming an anisotropic phase. In some embodiments, the assembling comprises polymerizing the plurality of gelators. In some embodiments, the assembling comprises aligning the plurality of gelators and then polymerizing the plurality of gelators. In some embodiments, the polymerizing and/or the aligning comprises applying an external field. In some embodiments, the external field comprises a magnetic field, an electric field, or an electromagnetic field. In some embodiments, the external field is applied in a direction that is sufficiently perpendicular to a surface of the substrate or a surface of the second substrate, if present. In some embodiments, the external field is applied in a direction that is sufficiently parallel to a surface of the substrate or a surface of the second substrate, if present. In some embodiments, the anisotropic phase comprises a plurality of gelators aligned in a direction that is sufficiently perpendicular to a surface of the substrate or to a surface of the second substrate, if present.

In some embodiments, the hydrogel comprises a network formed from a plurality of gelators, optionally wherein the plurality of gelators comprises a hydrogelator or a self-assembled gelator. In some embodiments, the pre-gel solution, if present, comprises a plurality of gelators. In some embodiments, the gelator comprises a ferromagnetic gelator, a paramagnetic gelator, a low molecular weight gelator optionally with a molecular weight of less than 3000 Daltons, an amphiphilic gelator, a nanofiber gelator, or a polymer-derived gelator. In some embodiments, the low molecular weight gelator comprises a peptide comprising an aromatic moiety, optionally wherein the peptide comprises a dipeptide or a tripeptide. In some embodiments, the aromatic moiety comprises naphthyl (Nap), fluorenylmethoxycarbonyl (Fmoc), fluorenyl, anthryl, phenanthryl, indenyl, or pyrenyl (Py). In some embodiments, the peptide comprises phenylalanine (Phe), lysine (Lys), tyrosine (Tyr), cyclohexylalanine (Cha), or a combination thereof. In some embodiments, the peptide comprises NapPhePhe, NapPhePhePhe, NapPhePhePheLys (Nap-SEQ ID NO: 1), NapPhePheLysLys (Nap-SEQ ID NO: 2), NapPhePhePheLysTyr, FmocPhe (Nap-SEQ ID NO: 3-Fmoc), FmocTyr, FmocPhePhe, or FmocPhePheLysLys (Fmoc-SEQ ID NO: 2).

In some embodiments, the nanofiber gelator comprises a silk nanofiber gelator or a peptide nanofiber gelator. In some embodiments, the silk nanofiber gelator comprises a dimension from about 10 to 100 nm, optionally wherein the dimension is a diameter or width. In some embodiments, the silk nanofiber gelator comprises silk or silk fibroin. In some embodiments, the peptide nanofiber gelator comprises a peptide sequence derived from silk fibroin or a fragment thereof. In some embodiments, the peptide sequence comprises GAGAGAGY (SEQ ID NO: 4), GAGAGY (SEQ ID NO: 5), GAGAGV (SEQ ID NO: 6), or GAGAGVGY (SEQ ID NO: 7). In some embodiments, the peptide sequence further comprises a hydrocarbon moiety, a fatty acid moiety, an ester of a fatty acid moiety, or an aromatic moiety. In some embodiments, the hydrocarbon moiety comprises lauryl (C12), capryl (C10), or caprylyl (C8); wherein the fatty acid moiety comprises lauric acid, caprylic acid, or capric acid; or wherein the ester of the fatty acid moiety comprises laurate, caprylate, or caprate. In some embodiments, the aromatic moiety comprises naphthyl (Nap), fluorenylmethoxycarbonyl (Fmoc), fluorenyl, anthryl, phenanthryl, indenyl, or pyrenyl (Py).

In some embodiments, the substrate, the first substrate, or the second substrate, if present, comprises a non-porous substrate comprising one of glass, silicon, poly-lysine coated material, nitrocellulose, polystyrene, cyclic olefin copolymers (COCs), cyclic olefin polymers (COPs), polypropylene, polyethylene, or polycarbonate.

In some embodiments, the biological sample is a fixed biological sample. In some embodiments, the fixed biological sample is a formalin-fixed paraffin-embedded biological sample, a PFA fixed biological sample, or an acetone fixed biological sample.

In some embodiments, any one of the methods provided herein further comprises staining and/or imaging the biological sample. In some embodiments, any one of the methods provided herein further comprises second strand synthesis. In some embodiments, any one of the methods provided herein further comprises sequencing. In some embodiments, the determining step comprises sequencing. In some embodiments, the sequencing comprises sequencing a sequence of the spatial barcode or a complement thereof; all or a portion of a sequence of the analyte from the biological sample, or a complement thereof; all or a part of a sequence of the nucleic acid of the biological sample bound to the capture domain, or a complement thereof.

Also provided herein are spatial arrays comprising: (a) a substrate; (b) a hydrogel comprising a plurality of anisotropic structures, wherein the plurality of anisotropic structures in the hydrogel comprises a polymeric network formed by assembling a plurality of gelators, thereby forming an anisotropic phase; and (c) a plurality of capture probes disposed between a surface of the substrate and a surface of the hydrogel, wherein at least one capture probe of the plurality of capture probes comprises: (i) a spatial barcode and (ii) a capture moiety.

Also provided herein are kits comprising: (a) a plurality of capture probes disposed between a surface of a substrate, wherein at least one capture probe of the plurality of capture probes comprises: (i) a spatial barcode and (ii) a capture moiety; and (b) a hydrogel or a pre-gel solution, wherein the hydrogel comprises a plurality of anisotropic structures in a polymeric network formed by assembling a plurality of gelators, or wherein the pre-gel solution comprises a plurality of gelators.

In some embodiments, any one of the kits described herein further comprises: (c) instructions for performing any one of the methods provided herein. In some embodiments, the substrate comprises any one of the spatial arrays provided herein. In some embodiments, the substrate is configured to provide a biological sample. In some embodiments, the biological sample is a fixed biological sample. In some embodiments, the fixed biological sample is a formalin-fixed paraffin-embedded biological sample, a PFA fixed biological sample, or an acetone fixed biological sample.

In some embodiments, any one of the kits described herein further comprises instructions for performing staining and/or imaging of the biological sample. In some embodiments, any one of the kits described herein further comprises one or more permeabilization reagents, reverse transcription (RT) reagents, second strand reagents, or amplification reagents. In some embodiments, any one of the kits described herein further comprises instructions for performing second strand synthesis or amplification.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “substantially complementary” used herein means that a first sequence is at least 50%, 55%, 60%, 65%, 70%, 75%, 80%, 85%, 90%, 95%, 97%, 98% or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, or that the two sequences hybridize under stringent hybridization conditions. Substantially complementary also means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations known to those skilled in the art.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows an exemplary workflow for performing templated capture and producing a ligation product.

FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe and an analyte capture agent.

FIG. 12 shows an exemplary schematic of an anisotropic structure on a substrate to reduce the lateral diffusion of analytes, or proxies thereof, from a biological sample.

FIG. 13 shows an exemplary schematic of a sandwiching process wherein a preprinted anisotropic structure is provided on a glass slide to mitigate analyte (e.g., transcript) mislocalization.

FIG. 14 shows an exemplary schematic of a sandwiching process wherein a hydrogel solution is added, and then external forces can be applied to form hydrogels with anisotropic structures.

FIG. 15A and FIG. 15B show exemplary schematics of an alignment of ferro- or paramagnetic nanofillers by applying a magnetic field (B₀) to generate an anisotropic hydrogel, wherein the hydrogel could either be prepared by a user (top schematic) or preprinted on the glass slide (bottom schematic).

FIG. 16A and FIG. 16B show exemplary schematics of an alignment of charged fibers by applying an electric field to generate an anisotropic hydrogel, wherein the hydrogel can be either pre-printed on the glass slide (top schematic) or prepared by having conductive slides to apply an electric field (E₀) within the slide sandwich (bottom schematic).

### DETAILED DESCRIPTION

The migration of analytes from a biological sample to a spatially arrayed slide is typically a passive process, wherein gravity causes analytes to migrate to a spatial array where they are hybridized to proximal capture domains of capture probes. However, passive migration also allows for mislocalization of analytes, wherein some of the analytes migrate and hybridize to non-proximal capture probe capture domains. Transcript mislocalization, or TML, can affect the resolution, sensitivity, and/or specificity of spatially arrayed gene expression data.

Described herein are methods and compositions that may be used to reduce TML when performing spatial analysis on a biological sample. Without wishing to be limited by mechanism, a system is proposed herein to provide a physical constraint (e.g., anisotropic structures) that limits movement of molecules during permeabilization of the biological sample, thereby controlling migration of target molecules in a downward manner so they interact with proximal capture probes instead of non-proximal capture probes, thereby minimizing transcript mislocalization. In some embodiments, the analyte can be a nucleic acid such as a mRNA or DNA, or the analytes can be an intermediate agent (e.g., a ligation product, such as any described herein) that is a proxy for a target analyte.

### A. Spatial Analysis Methods

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid)) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Patent Application Publication Nos. WO2018/091676, WO2020/176788, WO2017/144338, and WO2016/057552; Non-patent literature references Rodriques et al., Science 363(6434): 1463-1467, 2019; Lee et al., Nat. Protoc. 10(3): 442-458, 2015; Trejo et al., PLOS ONE 14(2): e0212031, 2019; Chen et al., Science 348(6233): aaa6090, 2015; Gao et al., BMC Biol. 15:50, 2017; and Gupta et al., Nature Biotechnol. 36:1197-1202, 2018; and the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022), both of which are available at the 10× Genomics Support Documentation website, and can be used herein in any combination, and each of which is incorporated herein by reference in its entirety. Further non-limiting aspects of spatial analysis methodologies and compositions are described herein.

Some general terminology that may be used in this disclosure can be found in Section (I)(b) of PCT Patent Application Publication No. WO2020/176788 and/or U.S. Patent Application Publication No. 2020/0277663, which is herein incorporated by reference. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Patent Application Publication No. WO2020/176788 and/or U.S. Patent Application Publication No. 2020/0277663, which is herein incorporated by reference. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples—which can be from different tissues or organisms—assembled on a single histologic slide. The TMA can therefore allow for high throughput analysis of multiple specimens at the same time. Tissue microarrays may be paraffin blocks produced by extracting cylindrical tissue cores from different paraffin donor blocks and re-embedding these tissue cores into a single recipient (microarray) block at defined array coordinates.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section (e.g., a fixed tissue section). In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plant, an insect, an arachnid, a nematode (e.g., Caenorhabditis elegans), a fungus, an amphibian, or a fish (e.g., zebrafish)). A biological sample can be obtained from a prokaryote such as a bacterium, e.g., Escherichia coli, Staphylococci or Mycoplasma pneumoniae; an archaeon; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example methanol. In some embodiments, instead of methanol, acetone, or an acetone-methanol mixture can be used. In some embodiments, the fixation is performed after sectioning. In some instances, when the biological sample is fixed using a fixative including an alcohol (e.g., methanol or acetone-methanol mixture), the biological sample is not decrosslinked afterward. In some preferred embodiments, the biological sample is fixed using a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample e.g., using acetone and/or alcohol (e.g., methanol, acetone-methanol) is performed while the sample is mounted on a substrate (e.g., glass slide, such as a positively charged glass slide).

In some embodiments, the biological sample, e.g., the tissue sample, is fixed, e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing, e.g., by formalin or PFA, the biological sample is dehydrated via sucrose gradient. In some instances, the fixed biological sample is treated with a sucrose gradient and then embedded in a matrix, e.g., OCT compound. In some instances, the fixed biological sample is not treated with a sucrose gradient, but rather is embedded in a matrix, e.g., OCT compound after fixation. In some embodiments when a fixed frozen tissue sample is treated with a sucrose gradient, the sample can be rehydrated using an ethanol gradient. In some embodiments, the PFA or formalin-fixed biological sample, which can be optionally dehydrated via sucrose gradient and/or embedded in OCT compound, is then frozen e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin-embedded. Thus, in preferred embodiments, a fixed frozen biological sample is not deparaffinized. In some embodiments, a fixed frozen biological sample is rehydrated in an ethanol gradient.

In some instances, the biological sample (e.g., a fixed frozen tissue sample) is treated with a citrate buffer. Citrate buffer can be used to decrosslink antigens and fixation medium in the biological sample for antigen retrieval. Thus, any suitable decrosslinking agent can be used in addition to or alternatively to citrate buffer. In some embodiments, for example, the biological sample (e.g., a fixed frozen tissue sample) is decrosslinked using TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, the sample is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, the sample can be rehydrated using an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, an acid, and a soluble organic compound that preserves morphology of biomolecules. PAXgene provides a two-reagent fixative system in which tissue is firstly fixed in a solution containing methanol and acetic acid, then stabilized in a solution containing ethanol. See, e.g., Ergin B. et al., J Proteome Res. 2010 Oct. 1; 9(10): 5188-96; Kap M. et al., PLOS One.; 6(11): e27704 (2011); and Mathieson W. et al., Am J Clin Pathol.; 146(1): 25-40 (2016), each of which is hereby incorporated by reference in its entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample, is fixed, for example in methanol, acetone, acetone-methanol, PFA, and/or PAXgene or is formalin-fixed and paraffin-embedded (FFPE). In some embodiments, the biological sample includes intact cells. In some embodiments, the biological sample is a cell pellet, e.g., a fixed cell pellet, e.g., an FFPE cell pellet. FFPE samples are used in some instances in the RNA-templated ligation (RTL) methods disclosed herein. A limitation of direct RNA capture for fixed samples is that the RNA integrity of fixed (e.g., FFPE) samples can be lower than of a fresh sample, thereby capturing RNA directly from fixed samples, e.g., by capture of a common sequence such as a poly(A) tail of an mRNA molecule, can be more difficult. However, by utilizing RTL probes that hybridize to RNA target sequences in the transcriptome, RNA analytes can be captured without requiring that both a poly(A) tail and target sequences remain intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Biological samples are also described in Section (I)(d) of PCT Patent Application Publication No. WO2020/176788 and/or U.S. Patent Application Publication No. 2020/0277663, which is herein incorporated by reference.

The following embodiments can be used with any of the methods described herein. In some embodiments, the biological sample (e.g., a fixed and/or stained biological sample) is imaged. In some embodiments, the biological sample is visualized or imaged using bright field microscopy. In some embodiments, the biological sample is visualized or imaged using fluorescence microscopy. The biological sample can be visualized or imaged using additional methods of visualization and imaging known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for analyzing captured analytes as disclosed herein to the biological sample.

In some embodiments, the methods include staining the biological sample. In some embodiments, the staining includes the use of hematoxylin and/or eosin. Non-limiting examples of stains include histological stains (e.g., hematoxylin and/or eosin) and immunological stains (e.g., fluorescent stains). In some embodiments, a biological sample can be stained using any number of biological stains, including but not limited to, acridine orange, Bismarck brown, carmine, coomassie blue, cresyl violet, DAPI (4′,6-diamidino-2-phenylindole), eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label, such as a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Patent Application Publication No. WO2020/176788 and/or U.S. Patent Application Publication No. 2020/0277663, which is herein incorporated by reference. Briefly, in any of the methods described herein, the method includes a step of permeabilizing the biological sample. For example, the biological sample can be permeabilized to facilitate transfer of extension products to the capture probes on the array. In some embodiments, the permeabilizing includes the use of an organic solvent (e.g., acetone, ethanol, or methanol), a detergent (e.g., saponin, Triton X-100™, Tween-20™, or sodium dodecyl sulfate (SDS)), an enzyme (e.g., an endopeptidase, an exopeptidase, or a protease), or a combination thereof. In some embodiments, the permeabilizing includes the use of an endopeptidase, a protease, SDS, polyethylene glycol tert-octylphenyl ether, polysorbate 80, polysorbate 20, N-lauroylsarcosine sodium salt solution, saponin, Triton X-100™, Tween-20™, or a combination thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, which is herein incorporated herein by reference.

Array-based spatial analysis methods can involve the transfer of one or more analytes or derivatives thereof from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Patent Application Publication No. WO2020/176788 and/or U.S. Patent Application Publication No. 2020/0277663, which is herein incorporated by reference. Generation of capture probes can be achieved by any appropriate method, including those described in Section (II)(d)(ii) of PCT Patent Application Publication No. WO2020/176788 and/or U.S. Patent Application Publication No. 2020/0277663, which is herein incorporated by reference.

In some instances, a capture probe and a nucleic acid analyte interaction (or any other nucleic acid to nucleic acid interaction) occurs because the sequences of the two nucleic acids are substantially complementary to one another. By “substantial,” “substantially,” and the like, two nucleic acid sequences can be complementary when at least 60% of the nucleotide residues of one nucleic acid sequence are complementary to nucleotide residues of the other nucleic acid sequence. The complementary residues within a particular complementary nucleic acid sequence need not always be contiguous with each other, but can be interrupted by one or more non-complementary residues within the complementary nucleic acid sequence. In some embodiments, at least 60%, but less than 100%, of the residues of one of the two complementary nucleic acid sequences are complementary to residues of the other nucleic acid sequence. In some embodiments, at least 70%, 80%, 90%, 95%, or 99% of the residues of one nucleic acid sequence are complementary to residues in the other nucleic acid sequence. Sequences are said to be “substantially complementary” when at least 60% (e.g., at least 70%, at least 80%, or at least 90%) of the residues of one nucleic acid sequence are complementary to residues of the other nucleic acid sequence. In some embodiments, the biological sample is mounted on a first substrate and the array of capture probes is on (e.g., affixed to) a second substrate. In this configuration, one or more analytes or analyte derivatives (e.g., intermediate agents, e.g., ligation products) are then released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described, e.g., in U.S. Patent Application Publication No. 2021/0189475 and PCT Patent Application Publication Nos. WO2021/252747 A1, WO2022/061152 A2, and WO2022/140028 A1, each of which is herein incorporated by reference.

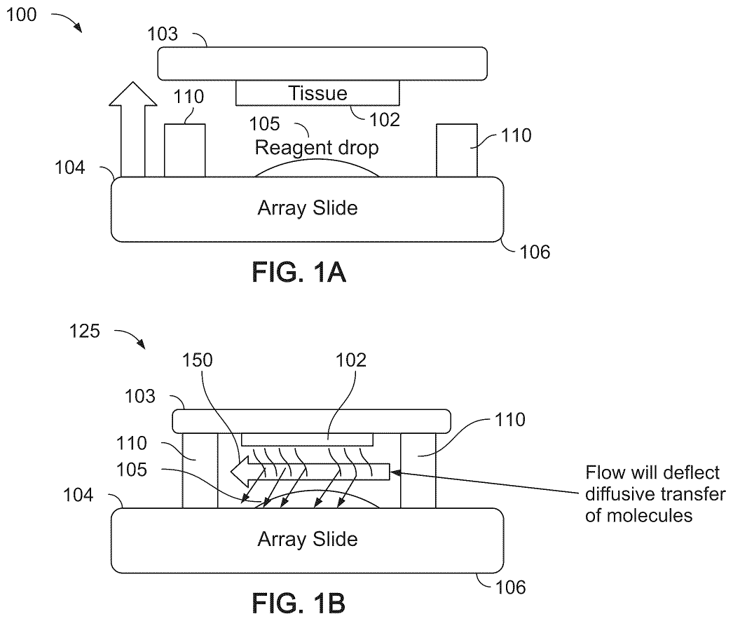

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A, a drop of liquid reagent (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents, e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. Herein, the reagent medium may also comprise one or more of a monovalent salt, a divalent salt, ethylene carbonate, and/or glycerol. In some embodiments, analytes (e.g., mRNA transcripts) and/or analyte derivatives (e.g., intermediate agents, e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent, e.g., ligation product) from the biological sample is performed actively (e.g., electrophoretic, by applying an electric field to promote migration). Exemplary methods of electrophoretic migration are described in WO2020/176788, and U.S. Patent Application Publication No. 2021/0189475, each of which is hereby incorporated by reference.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns (μm) and about 1 millimeters (mm), e.g., between about 2 μm and about 800 μm, between about 2 μm and about 700 μm, between about 2 μm and about 600 μm, between about 2 μm and about 500 μm, between about 2 μm and about 400 μm, between about 2 μm and about 300 μm, between about 2 μm and about 200 μm, between about 2 μm and about 100 μm, between about 2 μm and about 25 μm, or between about 2 μm and about 10 μm, measured in a direction orthogonal to the surface of the first substrate that supports the biological sample and the surface of the second substrate including the capture probes. In some instances, the separation distance is about 2, 3, 4, 5, 6, 7, 8, 9, 10, 11, 12, 13, 14, 15, 16, 17, 18, 19, 20, 21, 22, 23, 24, or 25 μm. In some embodiments, the separation distance is less than 50 μm. In some embodiments, the separation distance is less than 25 μm. In some embodiments, the separation distance is less than 20 μm. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents, e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents, e.g., ligation products) for spatial analysis. A partially or fully sealed chamber 150 resulting from the one or more spacers 110, the first substrate (e.g., slide 103), and the second substrate (e.g., slide 104) may reduce or prevent flow from undesirable movement (e.g., convective movement) of transcripts and/or molecules during the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., U.S. Patent Application Publication No. 2021/0189475, and PCT Patent Application Publication No. WO2022/061152 A2, each of which is incorporated by reference in its entirety.

In some embodiments, the sample holder can include a first member including a first retaining mechanism configured to retain a first substrate including a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

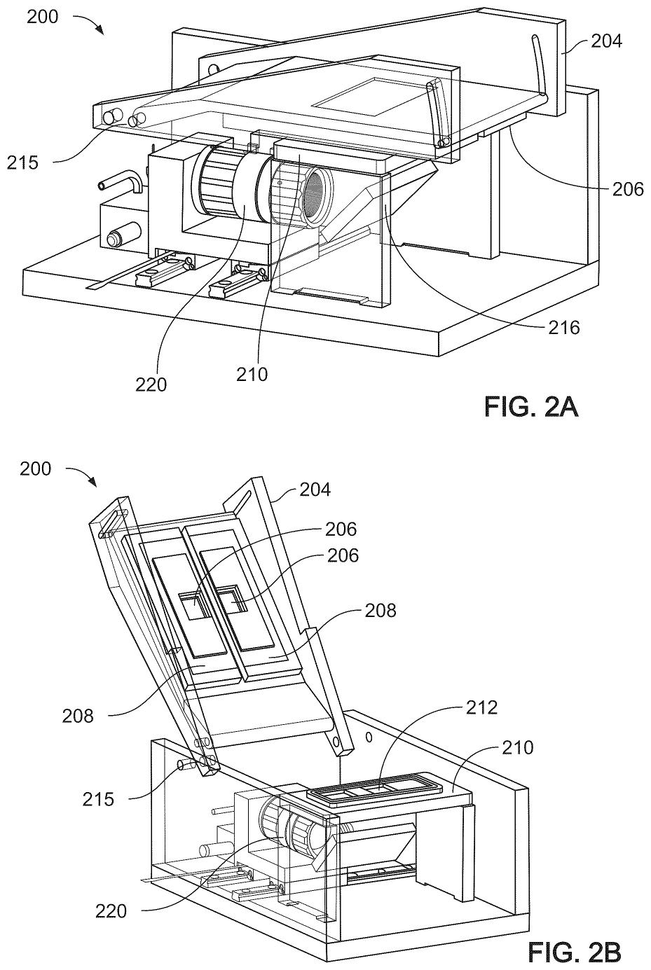

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents, e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

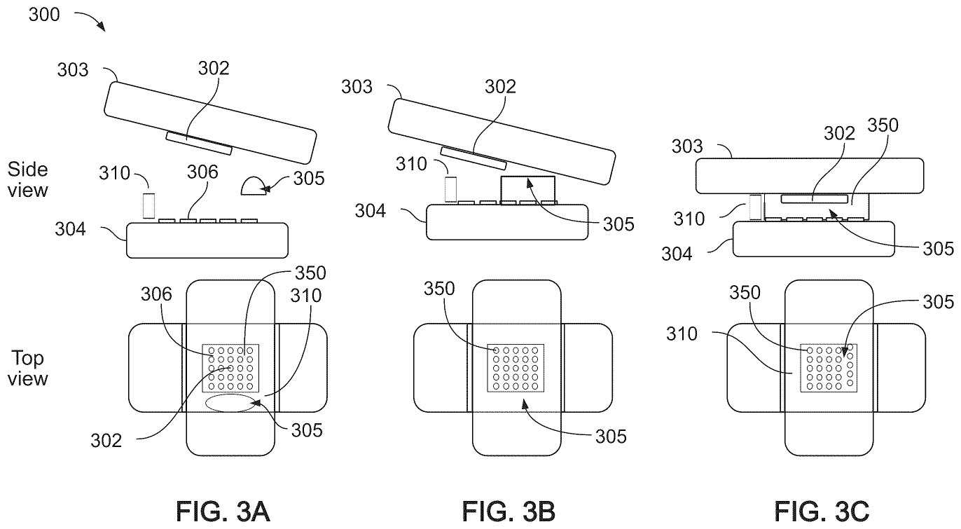

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., the slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right-hand side of side view, it should be understood that such depiction is not meant to be limiting as to the location of the reagent medium on the spacer.

FIG. 3B shows that as the first substrate lowers and/or as the second substrate rises, the dropped side of the first substrate (e.g., a side of the slide 303 angled toward the slide 304) may contact the reagent medium 305. The dropped side of the slide 303 may urge the reagent medium 305 toward the opposite direction (e.g., towards an opposite side of the spacer 310, towards an opposite side of the slide 303 relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate including the spacer 310, it should be understood that an exemplary angled closure workflow can include the second substrate angled over (superior to) the first substrate and the first substrate including the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

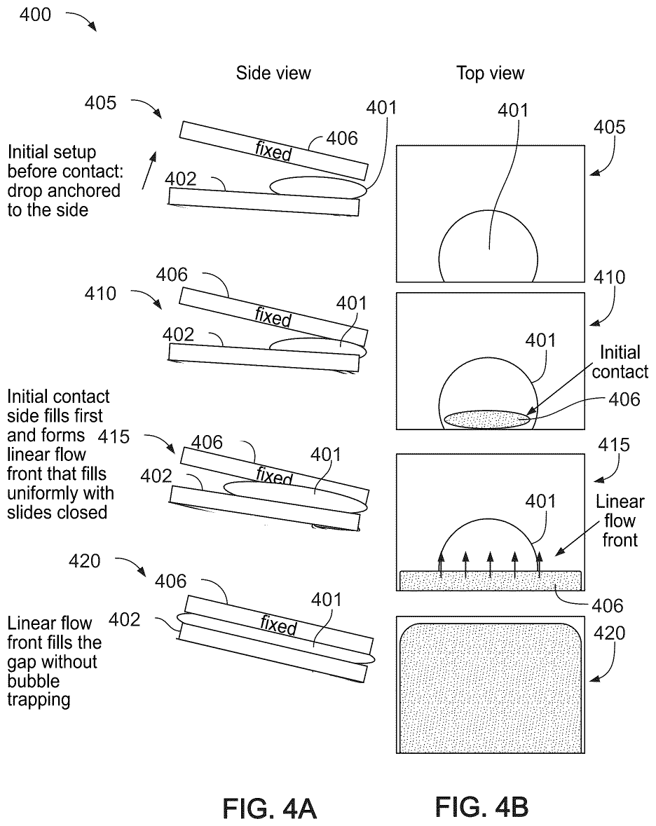

FIG. 4A is a side view of the angled closure workflow 400 in accordance with some exemplary implementations. FIG. 4B is a top view of the angled closure workflow 400 in accordance with some exemplary implementations. As shown at step 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills the gap between the two substrates 406 and 402 uniformly with the slides closed.

At step 415, the substrate 406 is further lowered toward the substrate 402 (or the substrate 402 is raised up toward the substrate 406) and the dropped side of the substrate 406 may contact and may urge the reagent medium toward the side opposite the dropped side, thereby creating a linear or low curvature flow front that may prevent or reduce bubble trapping between the substrates.

At step 420, the reagent medium 401 fills the gap between the substrate 406 and the substrate 402. The linear flow front of the liquid reagent may be formed by squeezing the reagent medium 401 volume along the contact side of the substrate 402 and/or the substrate 406. Additionally, capillary flow may also contribute to filling the gap area.

In some embodiments, the reagent medium (e.g., 105 in FIG. 1A) includes a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, or methanol), cross-linking agents (e.g., paraformaldehyde), detergents (e.g., saponin, Triton X-100™, Tween-20™, or SDS), and enzymes (e.g., trypsin or other proteases (e.g., Proteinase K)). In some embodiments, the detergent is an anionic detergent (e.g., SDS or N-lauroylsarcosine sodium salt solution).

In some embodiments, the reagent medium includes a lysis reagent. Lysis solutions can include ionic surfactants such as, for example, sarkosyl, and SDS. More generally, chemical lysis agents can include, without limitation, organic solvents, chelating agents, detergents, surfactants, and chaotropic agents. In some embodiments, the reagent medium includes a protease. Exemplary proteases include, e.g., pepsin, trypsin, elastase, and Proteinase K. In some embodiments, the reagent medium includes a nuclease. In some embodiments, the nuclease includes an RNase. In some embodiments, the RNase includes RNase A, RNase C, RNase H, and/or RNase I. In some embodiments, the reagent medium includes one or more of SDS or a sodium salt thereof, Proteinase K, pepsin, N-lauroylsarcosine, and RNase.

In some embodiments, the reagent medium includes polyethylene glycol (PEG). In some embodiments, the molecular weight of the PEG is from about 2K to about 16K. In some embodiments, the molecular weight of the PEG is about 2K, about 3K, about 4K, about 5K, about 6K, about 7K, about 8K, about 9K, about 10K, about 11K, about 12K, about 13K, about 14K, about 15K, or about 16K. In some embodiments, the PEG is present at a concentration from about 2% to about 25%, from about 4% to about 23%, from about 6% to about 21%, or from about 8% to about 20% (v/v).

In certain embodiments, a dried permeabilization reagent is applied or formed as a layer on the first substrate, the second substrate, or both prior to contacting the biological sample with the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Patent Application Publication No. WO2020/176788 and/or U.S. Patent Application Publication No. 2020/0277663 regarding extended capture probes, which is herein incorporated by reference). In some cases, capture probes may be configured to form ligation products with a template (e.g., a DNA or RNA template, such as an analyte or an intermediate agent, or portion thereof), thereby creating ligation products that serve as proxies for the template.

As used herein, an “extended capture probe” refers to a capture probe having additional nucleotides added to a terminus (e.g., a 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent bound to the capture domain of the capture probe. In some embodiments, the capture probe is extended using a reverse transcriptase. In some embodiments, the capture probe is extended using one or more DNA polymerases. In some embodiments, the extended capture probes include the sequence of the capture domain, the sequence of the spatial barcode of the capture probe, and the complementary sequence of the template used for extension of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II)(a) of PCT Patent Application Publication No. WO2020/176788 and/or U.S. Patent Application Publication No. 2020/0277663, which is herein incorporated by reference. Analysis of captured analytes (and/or intermediate agents or portions thereof), for example, including sample removal, extension of capture probes using the capture analyte as a template, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of PCT Patent Application Publication No. WO2020/176788 and/or U.S. Patent Application Publication No. 2020/0277663, which is herein incorporated by reference. Some quality control measures are described in Section (II)(h) of PCT Patent Application Publication No. WO2020/176788 and/or U.S. Patent Application Publication No. 2020/0277663, which is herein incorporated by reference.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660, each of which is herein incorporated by reference in its entirety.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up-regulated and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of PCT Patent Application Publication No. WO2020/176788 and/or U.S. Patent Application Publication No. 2020/0277663, which is herein incorporated by reference. Exemplary features and geometric attributes of an array can be found in Sections (II)(d)(i), (II)(d)(iii), and (II)(d)(iv) of PCT Patent Application Publication No. WO2020/176788 and/or U.S. Patent Application Publication No. 2020/0277663, which is herein incorporated by reference.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads or wells) including capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(c) of PCT Patent Application Publication No. WO2020/176788 and/or U.S. Patent Application Publication No. 2020/0277663, which is herein incorporated by reference.

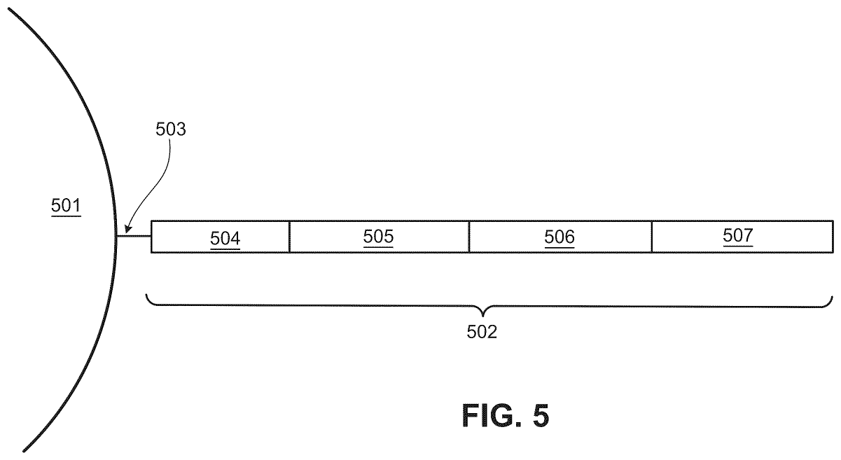

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that is useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to an analyte capture sequence present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

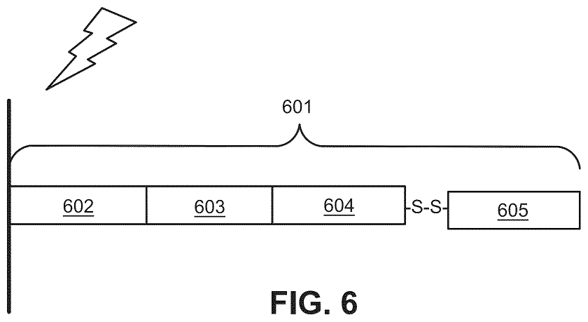

FIG. 6 is a schematic illustrating a cleavable capture probe, wherein the cleaved capture probe can enter into a non-permeabilized cell and bind to analytes within the cell. The capture probe 601 can contain a cleavage domain 602, a cell penetrating peptide 603, a reporter molecule 604, and a disulfide bond (—S—S—). 605 represents all other parts of a capture probe, for example, a spatial barcode and a capture domain.

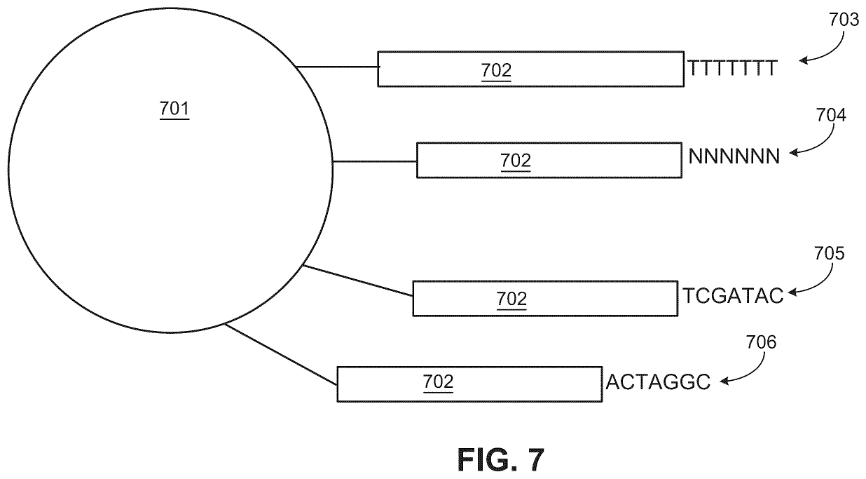

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may include four different types of spatially-barcoded capture probes, each type of spatially-barcoded capture probe possessing the spatial barcode 702. One type of capture probe associated with the feature can include the spatial barcode 702 in combination with a poly(T) capture domain 703, designed to capture mRNA target analytes. A second type of capture probe associated with the feature can include the spatial barcode 702 in combination with a random N-mer capture domain 704 for gDNA analysis. A third type of capture probe associated with the feature can include the spatial barcode 702 in combination with a capture domain complementary to the analyte capture agent of interest 705. A fourth type of capture probe associated with the feature can include the spatial barcode 702 in combination with a capture probe that can bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and/or metabolites, and gDNA; (b) mRNA, accessible chromatin (e.g., ATAC-seq, DNase-seq, and/or MNase-seq), cell surface or intracellular proteins and/or metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V (D) J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequence 504 are common to all of the probes attached to a given feature. In some embodiments, the UMI sequence 506 of a capture probe attached to a given feature is different from the UMI sequence of a different capture probe attached to the given feature.

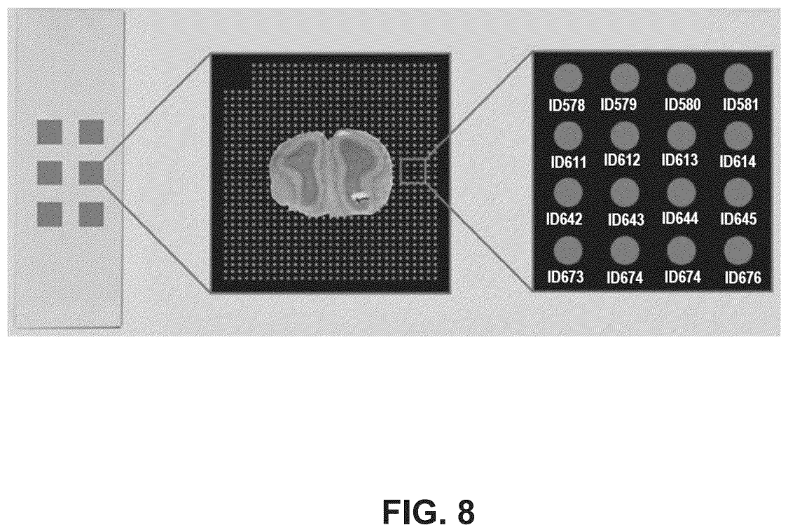

FIG. 8 depicts an exemplary arrangement of barcoded features within an array. From left to right, FIG. 8 shows (left) a slide including six spatially-barcoded arrays, (center) an enlarged schematic of one of the six spatially-barcoded arrays, showing a grid of barcoded features in relation to a biological sample, and (right) an enlarged schematic of one section of an array, showing the specific identification of multiple features within the array (e.g., labelled as ID578, ID579, ID580, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Patent Application Publication No. WO2020/176788 and/or U.S. Patent Application Publication No. 2020/0277663, which is herein incorporated by reference.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell or cell nucleus in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells or cell nuclei in a biological sample) for use in spatial analysis. In some embodiments, after attaching and/or introducing a molecule having a barcode to a biological sample, the biological sample can be physically separated (e.g., dissociated) into single cells, single cell nuclei, or cell groups for analysis. Some such methods of spatial analysis are described in Section (III) of PCT Patent Application Publication No. WO2020/176788 and/or U.S. Patent Application Publication No. 2020/0277663, which is herein incorporated by reference.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128, which is herein incorporated by reference. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence or a non-homopolymeric sequence). After hybridization to the analyte, a ligase (e.g., a T4 RNA ligase (Rnl2), a PBCV-1 DNA Ligase or Chlorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides can create a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location, and optionally, the abundance of the analyte in the biological sample.

In some instances, one or both of the oligonucleotides may hybridize to genomic DNA (gDNA) which can lead to false positive sequencing data from ligation events on gDNA (off target) in addition to the desired (on target) ligation events on target nucleic acids, (e.g., mRNA). Thus, in some embodiments, the disclosed methods can include contacting the biological sample with a deoxyribonuclease (DNase). The DNase can be an endonuclease or exonuclease. In some embodiments, the DNase digests single-stranded and/or double-stranded DNA. Suitable DNases include, without limitation, a DNase I and a DNase II. Use of a DNase as described can mitigate false positive sequencing data from off target gDNA ligation events.

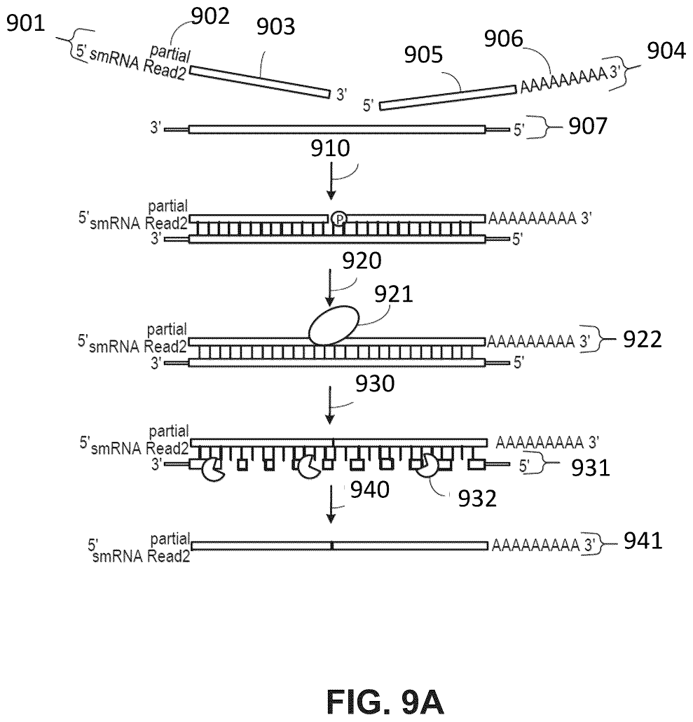

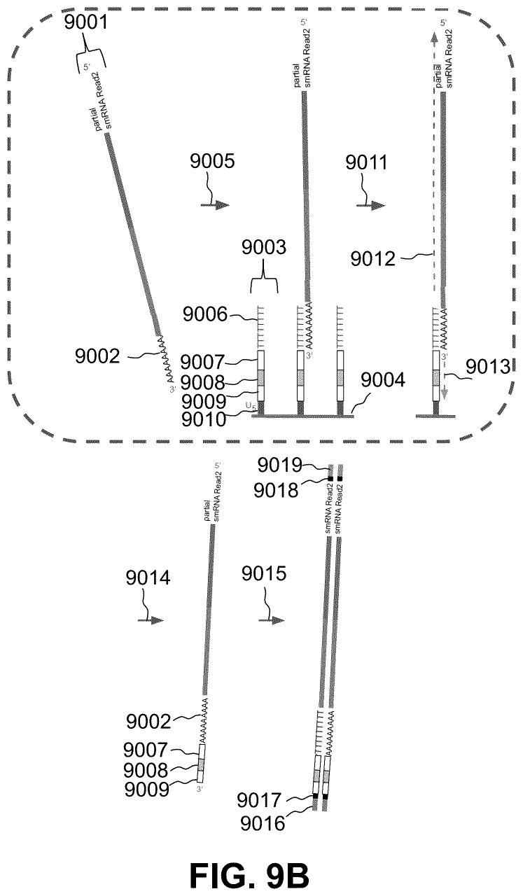

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly(A) sequence) 906, the first probe 901 and the second probe 904 hybridize 910 to an analyte 907. A ligase 921 ligates 920 the first probe 901 to the second probe 904, thereby generating a ligation product 922. The ligation product 922 is then released 930 from the analyte 931 by digesting the analyte 907 using an endoribonuclease 932. The sample is permeabilized 940 and the ligation product 941 is able to hybridize to a capture probe on the substrate. Methods and compositions for spatial detection using templated ligation have been described in PCT Patent Application Publication No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product 9001 binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily bind to target analytes (i.e., compared to no permeabilization). In some embodiments, reverse transcription (RT) reagents can be added to permeabilize biological samples. Incubation with the RT reagents can be used to extend the capture probes 9011 to produce spatially-barcoded full-length cDNA 9012 and 9013 from the captured analytes (e.g., polyadenylated mRNA). Second strand reagents (e.g., second strand primers, enzymes, etc.) can be added to the biological sample to initiate second strand synthesis.

In some embodiments, methods provided herein include permeabilization of the biological sample such that the capture probe can more easily capture the ligation products (i.e., compared to no permeabilization). In some embodiments, polymerization (e.g., reverse transcription (RT)) reagents can be added to permeabilized biological samples. Incubation with the RT reagents can be used to extend the capture probes 9011 to produce spatially-barcoded full-length cDNA 9012 and 9013 from the captured ligation products (e.g., polyadenylated ligation products).

In some embodiments, the extended ligation products can be denatured 9014, released from the capture probe, and transferred (e.g., to a clean tube) for amplification, and/or library construction. The spatially-barcoded ligation products can be amplified 9015 via PCR prior to library construction. P5 9016 and P7 9019 sequences can be used for sequencing, while i5 9017 and i7 9018 sequences can be used as sample indexes. The amplicons can then be sequenced using paired-end sequencing using TruSeq Read 1 and TruSeq Read 2 as sequencing primer sites for Illumina sequencers. Other sequencing systems can be used, all that is required is the sequences specific to a particular instrument and workflow is incorporated into the sequencing libraries.

In some embodiments, in addition to the detection of genetic variants in a biological sample, detection of one or more other analytes (e.g., protein analytes) can be performed, either sequentially or concurrently, using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b) (ix) of PCT Patent Application Publication No. WO2020/176788 and/or Section (II)(b)(viii) U.S. Patent Application Publication No. 2020/0277663, which is herein incorporated by reference.

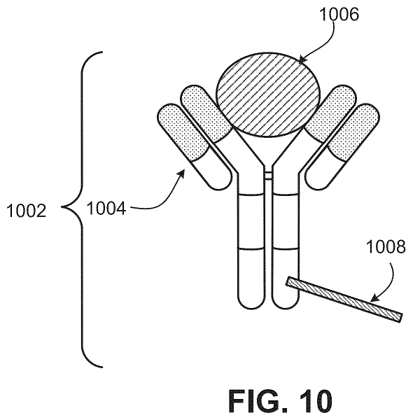

FIG. 10 is a schematic diagram of an exemplary analyte capture agent 1002 comprised of an analyte binding moiety 1004 and an analyte binding moiety barcode domain 1008. The analyte binding moiety 1004 is a molecule capable of binding to an analyte 1006 and the analyte capture agent 1002 is capable of interacting with a spatially-barcoded capture probe, e.g., on an array. The analyte binding moiety 1004 can bind to the analyte 1006 with high affinity and/or with high specificity. The analyte capture agent 1002 can include: (i) an analyte binding moiety barcode domain 1008, which serves to identify the analyte binding moiety, and (ii) a capture domain, which can hybridize to at least a portion or an entirety of a capture domain of a capture probe. The analyte binding moiety 1004 can include a polypeptide and/or an aptamer. The analyte binding moiety 1004 can include an antibody or antibody fragment (e.g., an antigen binding fragment).

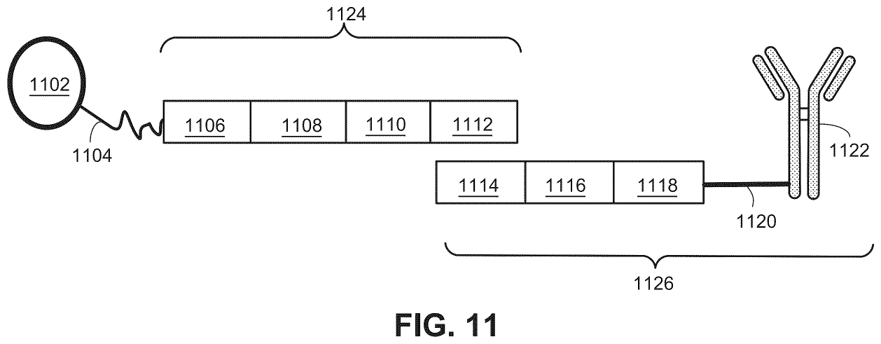

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequence 1106 and a UMI 1110, as described elsewhere herein. The capture probe can be affixed 1104 to a feature such as a bead 1102. The capture probe 1124 can also include a capture domain 1112 that is capable of binding to an analyte capture agent 1126. The analyte binding moiety barcode domain of the analyte capture agent 1126 can include a functional sequence 1118, analyte binding moiety barcode 1116, and an analyte capture sequence 1114 that is capable of binding (e.g., hybridizing) to the capture domain 1112 of the capture probe 1124. The analyte capture agent 1126 can also include a linker 1120 that allows the analyte binding moiety barcode domain (e.g., including the functional sequence 1118, analyte binding moiety barcode 1116, and analyte capture sequence 1114) to couple to the analyte binding moiety 1122. In some embodiments, the linker 1120 is a cleavable linker. In some embodiments, the cleavable linker is a photo-cleavable linker, a UV-cleavable linker, chemical-cleavable linker, thermal-cleavable linker, or an enzyme cleavable linker. In some instances, the cleavable linker is a disulfide linker. A disulfide linker can be cleaved by use of a reducing agent, such as dithiothreitol (DTT), beta-mercaptoethanol (BME), or tris(2-carboxyethyl) phosphine (TCEP).

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that each spatial barcode is uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location or a fiducial marker) of the array. Accordingly, each feature location has an “address” or location in the coordinate space of the array.

Some exemplary spatial analysis workflows are described in the Exemplary Embodiments section of PCT Patent Application Publication No. WO2020/176788 and/or U.S. Patent Application Publication No. 2020/0277663, which is herein incorporated by reference. See, e.g., the Exemplary embodiment starting with “In some non-limiting examples of the workflows described herein, the sample can be immersed . . . ” of PCT Patent Application Publication No. WO2020/176788 and/or U.S. Patent Application Publication No. 2020/0277663, which is herein incorporated by reference. See also, e.g., the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022), each of which is herein incorporated by reference in its entirety.

In some embodiments, spatial analysis can be performed using dedicated hardware and/or software, such as any of the systems described in Sections (II)(c) (ii) and/or (V) of PCT Patent Application Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Patent Application Publication No. WO2020/123320, which is herein incorporated by reference.

Suitable systems for performing spatial analysis can include components such as a chamber (e.g., a flow cell or a scalable, fluid-tight chamber) for containing a biological sample. The biological sample can be mounted, for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid-state storage medium such as, but not limited to, a magnetic, optical, or other solid state, persistent, writeable, and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD or CMOS) used to capture images. The systems can also optionally include one or more light sources (e.g., LED-based, diode-based, or lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Patent Application Publication No. WO2021/102003 and/or U.S. Patent Application Publication No. 2021/0150707, each of which is incorporated herein by reference in its entirety.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-dimensional and/or three-dimensional map of the analyte presence and/or level are described in PCT Patent Application Publication No. WO2020/053655 and spatial analysis methods are generally described in PCT Patent Application Publication No. WO2021/102039 and/or U.S. Patent Application Publication No. 2021/0155982, each of which is incorporated herein by reference in its entirety.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Patent Application Publication Nos. WO2020/123320, WO2021/102005, and/or U.S. Patent Application Publication No. 2021/0158522, each of which is incorporated herein by reference in its entirety. Fiducial markers can be used as a point of reference or measurement scale for alignment (e.g., to align a sample and an array, to align two substrates, or to determine a location of a sample or array on a substrate relative to a fiducial marker) and/or for quantitative measurements of sizes and/or distances.

### B. Methods of Reducing Transcript Mislocalization of a Target Analyte

Spatial transcriptomics would benefit from methods that could minimize target analyte mislocalization. For example, analytes (e.g., target nucleic acids or proxies thereof) typically migrate vertically to the substrate where they are captured by the capture domain of a capture probe. Rather than vertical movement, analytes could also migrate in a non-vertical manner (e.g., a horizontal movement), thereby leading to analyte mislocalization, including transcript mislocalization (e.g., due to diffusion, flow, convection). This in turn can result in incorrect registration of spatial information, which can lead to incorrect detection of spatial gene expression and cell type identification in spatial data. As such, reducing transcript mislocalization would have a positive impact on the quality of spatial data.

Accordingly, in some embodiments, the methods herein provides a physical constraint (e.g., by way of an anisotropic structure) that limits movement of molecules in a first direction (e.g., a first direction that is substantially parallel to a surface of the substrate), while facilitating or promoting movement of molecules in a second direction (e.g., a second direction that is substantially perpendicular to a surface of the substrate). Such methods can be implemented before or during other methods involving capturing analytes, such as transcripts or probes (e.g., methods including permeabilization of the biological sample). For example, when a tissue sample is digested, the anisotropic structure remains intact to regulate flow or movement of molecules (e.g., transcripts or probes) towards the substrate, thereby minimizing transcript mislocalization. In some embodiments, the analyte can be an intermediate agent (e.g., a ligation product, such as any described herein), which can optionally serve as a proxy for a target analyte.

Provided herein are methods for determining the location of an analyte in a biological sample, the methods including: (a) providing the biological sample on a substrate, wherein the substrate comprises a plurality of capture probes, and wherein a hydrogel comprising a plurality of anisotropic structures is disposed between the biological sample and a surface of the substrate comprising the plurality of capture probes; (b) hybridizing or contacting the analyte, or a proxy thereof, with at least one capture probe of the plurality of capture probes, wherein the at least one capture probe comprises a capture domain and a spatial barcode; and (c) determining (i) the spatial barcode or a complement thereof, and (ii) all or a portion of the analyte from the biological sample, and using the determined sequences of (i) and (ii) to determine the location of the analyte in the biological sample.

As used herein, a “capture probe” refers to any molecule capable of capturing (directly or indirectly) and/or labelling an analyte (e.g., an analyte of interest or a proxy thereof) in a biological sample. In some embodiments, the capture probe is a nucleic acid or a polypeptide. In some embodiments, the capture probe includes a barcode (e.g., a spatial barcode) and a capture domain. In some embodiments, a capture probe can include one or more of a unique molecular identifier (UMI), a cleavage domain, and/or a functional sequence (e.g., a primer-binding site, a sequencing domain such as for next-generation sequencing (NGS)).

In some embodiments, a capture probe includes a capture domain and a spatial barcode. In some embodiments, the capture domain interacts with the analyte, or the proxy thereof. The capture domain can be configured to capture any analyte of interest. In some embodiments, the analyte of interest is an intermediate agent. In some embodiments, the intermediate agent is a ligation product, for example, which is a proxy for a target analyte. In some embodiments, the analyte of interest is a target sequence. In some embodiments, the target sequence can include a synthetic or natural sequence. In some embodiments, the target sequence is a DNA or an RNA (e.g., a mRNA). In some embodiments, the spatial barcode comprises a sequence unique to the location or feature on the substrate where the capture probe is positioned that can be used to identify the location of the target analyte on the substrate.

In some embodiments, the biological sample is permeabilized to allow the analyte, or the proxy thereof, in or on the biological sample to interact with the at least one capture probe of the plurality of capture probes. In some embodiments, any one of the methods described herein further comprises (e.g., before or during the hybridizing or contacting step): permeabilizing the biological sample to allow the analyte, or the proxy thereof, in or on the biological sample to interact with the at least one capture probe of the plurality of capture probes.

Also provided herein are methods for determining the location of an analyte in a biological sample, the methods including: (a) providing the biological sample on a first substrate; (b) aligning the first substrate with a second substrate comprising an array and a hydrogel, such that at least a portion of the biological sample is aligned with at least a portion of the array, and such that at least a portion of the hydrogel is disposed between the biological sample and the second substrate, wherein the array comprises a plurality of capture probes, and wherein the hydrogel comprises a plurality of anisotropic structures; (c) when the biological sample is aligned with at least a portion of the array and at least a portion of the hydrogel, hybridizing or contacting the analyte, or a proxy thereof, with at least one capture probe of the plurality of capture probes, wherein the at least one capture probe comprises a capture domain and a spatial barcode; and (d) determining (i) the spatial barcode or a complement thereof, and (ii) all or a portion of the analyte from the biological sample, and using the determined sequences of (i) and (ii) to determine the location of the analyte in the biological sample.

Also provided herein are methods for determining the location of an analyte in a biological sample, the methods including: (a) providing the biological sample on a first substrate; (b) aligning the first substrate with a second substrate comprising an array, such that at least a portion of the biological sample is aligned with at least a portion of the array, and such that at least a portion of a pre-gel solution is disposed between the biological sample and the second substrate, wherein the array comprises a plurality of capture probes; (c) applying an external field to the pre-gel solution and optionally applying a reagent to promote gelation of the pre-gel solution, thereby providing a hydrogel comprising a plurality of anisotropic structures; (d) when the biological sample is aligned with at least a portion of the array and at least a portion of the hydrogel, hybridizing or contacting the analyte, or a proxy thereof, with at least one capture probe of the plurality of capture probes, wherein the at least one capture probe comprises a capture domain and a spatial barcode; and (c) determining (i) the spatial barcode or a complement thereof, and (ii) all or a portion of the analyte from the biological sample, and using the determined sequences of (i) and (ii) to determine the location of the analyte in the biological sample.

In some embodiments, the methods further include (e.g., before or during the hybridizing or contacting step): permeabilizing the biological sample to allow the analyte, or the proxy thereof, in the biological sample to interact with at least one capture probe of the plurality of capture probes.

Also provided herein are methods for determining the location of a nucleic acid in a biological sample, the methods including: (a) contacting the biological sample with a substrate, wherein the substrate comprises: (i) a plurality of capture probes and (ii) a hydrogel comprising plurality of anisotropic structures, such that the hydrogel is disposed between the biological sample and a surface of the substrate comprising the plurality of capture probes; (b) hybridizing or contacting the nucleic acid of the biological sample, or a proxy thereof, with at least one capture probe of the plurality of capture probes, wherein the at least one capture probe comprises a capture domain and a spatial barcode; (c) extending a 3′ end of the at least one capture probe using the nucleic acid of the biological sample, or the proxy thereof, bound to the capture domain as a template to generate an extended capture probe; and (d) determining (i) all or a part of a nucleotide sequence of the nucleic acid of the biological sample bound to the capture domain, or a complement thereof, and (ii) a nucleotide sequence of the spatial barcode, or a complement thereof, and using the determined nucleotide sequences of (i) and (ii) to determine the location of the nucleic acid in the biological sample.

Also provided herein are methods for determining the location of a nucleic acid in a biological sample, the methods including: (a) contacting the biological sample with a substrate, wherein the substrate comprises a plurality of capture probes; (b) contacting the biological sample with a pre-gel solution that is disposed on a surface of the substrate; (c) applying an external field to the pre-gel solution and optionally applying a reagent to promote gelation of the pre-gel solution, thereby providing a hydrogel comprising a plurality of anisotropic structures; (d) hybridizing or contacting the nucleic acid of the biological sample, or a proxy thereof, with at least one capture probe of the plurality of capture probes, wherein the at least one capture probe comprises a capture domain and a spatial barcode; (e) extending a 3′ end of the at least one capture probe using the nucleic acid of the biological sample, or the proxy thereof, bound to the capture domain as a template to generate an extended capture probe; and (f) determining (i) all or a part of a nucleotide sequence of the nucleic acid of the biological sample bound to the capture domain, or a complement thereof, and (ii) a nucleotide sequence of the spatial barcode, or a complement thereof, and using the determined nucleotide sequences of (i) and (ii) to determine the location of the nucleic acid in the biological sample.

In some embodiments, the methods further include (e.g., before or during the hybridizing or contacting step): permeabilizing the biological sample to allow the nucleic acid in the biological sample, or the proxy thereof, to interact with the at least one capture probe of the plurality of capture probes. In some embodiments, the method further includes generating a complement of the extended capture probe. In some embodiments, the method further includes releasing the complement of the extended capture probe and optionally amplifying the complement of the extended capture probe. In some embodiments, the nucleic acid is DNA. In some embodiments, the nucleic acid is RNA.

Also provided herein are methods for mitigating mislocalization of analytes captured on a spatial array, the methods including: (a) providing the biological sample on a substrate, wherein the substrate comprises a spatial array comprising a plurality of capture probes, and wherein a hydrogel comprising a plurality of anisotropic structures is disposed between the biological sample and the spatial array; and (b) hybridizing or capturing an analyte, or the proxy thereof, using at least one capture probe of the plurality of capture probes, wherein the at least one capture probe comprises a capture domain and a spatial barcode, thereby mitigating mislocalization of the captured analyte, or the captured proxy thereof, and its subsequent mislocalization on the spatial array.

In some embodiments, the hybridizing or capturing step comprises migration of the captured analyte, or the captured proxy thereof, wherein the migration comprises vertical migration between the biological sample and the spatial array. In some embodiments, the methods further include: determining (i) the spatial barcode or a complement thereof, and (ii) all or a portion of the sequence of the analyte from the biological sample.

C. Hydrogels with Anisotropic Structures

In some embodiments, the method described here can include providing a biological sample on a substrate, wherein the substrate comprises a plurality of capture probes, and wherein a hydrogel comprising a plurality of anisotropic structures is disposed between the biological sample and a surface of the substrate comprising the plurality of capture probes.

As used herein, a “hydrogel” can refer to a gel in which the swelling agent is water. In turn, the gel may be composed of a polymeric network. In some embodiments, the hydrogel can include a cross-linked three-dimensional (3D) network of hydrophilic polymer chains. Hydrogels can be synthesized from chain polymerization of monomers (e.g., multifunctional macromolecular monomers), wherein the hydrogel can contain a high volume of water while being structurally stable. Such stability can be provided by chemical or physical interactions (including covalent or non-covalent interactions), such as by cross-linking of polymer chains or by induced gelation of monomers by an external stimulus (e.g., pH, ionic concentration, temperature, and the like). In some embodiments, hydrogels are synthesized using a monomer, an optional cross-linking chemical, and specific reaction conditions. In some examples, a monomer can include a reactive group that allows for polymerization. In other examples, a monomer can be used with another co-monomer and/or a cross-linking chemical during polymerization. For example, based on the methods of synthesis, hydrogels may be classified as a copolymer, a semi-interpenetrating network (semi-IPN) homopolymer, or an interpenetrating network (IPN). In some embodiments, homopolymers have only one type of monomer in their structure and, based on the monomer's nature and the polymerization process, they may have a cross-linked structure. In some embodiments, copolymeric hydrogels can have a plurality of monomers, and at least one monomer in copolymeric hydrogels is hydrophilic. In some embodiments, a hydrogel can include a salt-triggered (e.g., a divalent salt-triggered, such as Ca²⁺ or Mg²⁺ salt-triggered) naphthalene conjugated short peptide (NapFF) gel or an acidifier-triggered (e.g., glucose-delta-lactone (GdL)-triggered) NapFF gel.

Hydrogels can include other solvents that are not water, such as in an organogel in which the swelling agent is an organic solvent (e.g., an ether, an alkane, an alcohol, or mixtures thereof). Hydrogels may be present in other forms, such as a xerogel or an aerogel.

In some embodiments, a hydrogel can include hydrogel subunits. A “hydrogel subunit” can be a hydrophilic monomer, a molecular precursor, or a polymer that can be polymerized (e.g., cross-linked) to form a hydrogel (e.g., a 3D hydrogel network). The hydrogel subunits can include any convenient hydrogel subunits, such as, but not limited to, acrylamide and derivatives thereof (e.g., alkyl acrylamide), bis-acrylamide and derivatives thereof (e.g., N,N-alkylene bis-acrylamide, such as N,N-methylenebisacrylamide), acrylate and derivatives thereof (e.g., sodium acrylate or alkyl acrylate), methacrylate and derivatives thereof (e.g., alkyl methacrylate or methacryloyl), bis-acrylate and derivatives thereof, polyacrylamide and derivatives thereof, poly(ethylene glycol) and derivatives thereof (e.g., PEG-acrylate (PEG-DA), PEG methacrylate (PEGMA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, heparin, fibrin, alginate, glutaraldehyde, protein polymers, methylcellulose, and the like, or combinations thereof. For any compounds described herein (e.g., monomers, catalysts, etc.), salt forms may be employed (e.g., sodium salts, hydrochloride salts, and the like), and ionic forms may be employed (e.g., anionic or cationic forms).

As used herein, an “anisotropic structure” refers to a material having direction- and dimension-dependent chemical and/or physical properties. In some embodiments, anisotropic structures can be formed into a one-dimensional (1D), two-dimensional (2D), or three-dimensional (3D) structure. Assembly of anisotropic structures into the 2D or 3D structures may largely depend on their shape, surface properties, charge, polarizability, magnetic dipole moment, or mass of the anisotropic structure. Examples of anisotropic structures can include, but are not limited to, nanorods, nanowires, nanotubes, nanofillers (e.g., ferro- or paramagnetic nanofillers), nanofibers (e.g., silk nanofibers), nanopolymers (e.g., linear polymers), and nanoneedles. In some embodiments, an anisotropic structure can be formed within a hydrogel. In some embodiments, an anisotropic structure can be used to reduce analyte mislocalization, wherein the anisotropic structure reduces the lateral diffusion of analytes. In some embodiments, a method described herein can include providing a biological sample on a substrate, wherein the substrate comprises a plurality of capture probes, and wherein a hydrogel comprising a plurality of anisotropic structures is disposed between the biological sample and a surface of the substrate. In some embodiments, a method described herein can include providing a biological sample on a first substrate, providing a second substrate comprising a plurality of capture probes, and wherein a hydrogel comprising a plurality of anisotropic structures is disposed between the biological sample on the first substrate and the second substrate.

In some embodiments, an anisotropic structure can be formed on a substrate prior to contacting a biological sample on the substrate. In some embodiments, the providing a biological sample on a substrate can include: (i) providing an initial substrate comprising the plurality of capture probes and the hydrogel comprising the plurality of anisotropic structures; and (ii) contacting the biological sample with the hydrogel, thereby providing the biological sample on the substrate. In some embodiments, the hydrogel comprising the plurality of anisotropic structures is prepared by (i) applying an external field to a pre-gel solution comprising a plurality of gelators, optionally wherein the pre-gel solution is disposed on a surface of the initial substrate, and (ii) optionally applying a reagent to promote gelation of the pre-gel solution, thereby providing the hydrogel comprising the plurality of anisotropic structures.

As used herein, a “gelator” refers to a molecule or compound that forms two-dimensional (2D) or three-dimensional (3D) networks in a given solvent, either through covalent cross-linking or noncovalent intermolecular interactions, resulting in the formation of a molecular gel (e.g., hydrogels). In some embodiments, a plurality of gelators (e.g., dipeptide-derived gelator) can form a hydrogel by adding an additional gelling reagent. In some embodiments, a plurality of gelators (e.g., silk-derived nanofiber gelator) can form a hydrogel without an additional gelling reagent.

In some embodiments, a hydrogel having anisotropic structures can include a polymeric network in which gelators are assembled into structures, which in turn provide direction- and dimension-dependent properties. For example, the gelators can form fibrillar structures that are aligned in a certain direction. In some embodiments, the fibrillar structure has a major dimension (e.g., a length) that is aligned substantially perpendicular to or parallel to the direction of an externally applied field. In some embodiments, the fibrillar structure has a major dimension (e.g., a length) that is aligned substantially perpendicular to or parallel to a surface of the substrate. Such fibrillar structures can be provided within the entire hydrogel or within a portion of the hydrogel (e.g., a portion in proximity to the biological sample or a portion in proximity to the substrate).

In some embodiments, the hydrogel comprises a network formed from a plurality of gelators (e.g., a hydrogelator or a self-assembled gelator). In some embodiments, the pre-gel solution, if present, comprises a plurality of gelators. In some embodiments, the gelator comprises a ferromagnetic gelator, a paramagnetic gelator, a low molecular weight gelator (e.g., a molecular weight of less than 3000 Daltons), an amphiphilic gelator, a nanofiber gelator, or a polymer-derived gelator. In some embodiments, a ferro- or paramagnetic gelator is a material that can be aligned in a magnetic field. In some embodiments, an amphiphilic gelator is a material containing hydrophobic and hydrophilic units. In some embodiments, a nanofiber gelator composites nanofiber in the hydrogel network. In some embodiments, a polymer-derived gelator is a hydrogel that composites polymers such as homopolymers and diblock copolymers. In some embodiments, the low molecular weight gelator comprises a peptide (e.g., a dipeptide, tripeptide, and the like) comprising an aromatic moiety. In some embodiments, the aromatic moiety comprises naphthyl (Nap), fluorenylmethoxycarbonyl (Fmoc), fluorenyl, anthryl, phenanthryl, indenyl, or pyrenyl (Py). In some embodiments, the peptide comprises phenylalanine (Phe), lysine (Lys), tyrosine (Tyr), cyclohexylalanine (Cha), or a combination thereof. In some embodiments, the peptide comprises NapPhePhe (NapFF), NapPhePhePhe (NapFFF), NapPhePhePheLys (NapFFFK) (Nap-SEQ ID NO: 1), NapPhePheLysLys (NapFFKK) (Nap-SEQ ID NO: 2), NapPhePhePheLysTyr (NapFFFKY) (Nap-SEQ ID NO: 3-Fmoc), FmocPhe (FmocF), FmocTyr (FmocY), FmocPhePhe (FmocFF), or FmocPhePheLysLys (FmocFFKK) (Fmoc-SEQ ID NO: 2).

In some embodiments, the nanofiber gelator comprises a silk nanofiber gelator or a peptide nanofiber gelator. In some embodiments, the silk nanofiber gelator comprises a dimension (e.g., diameter, width, and the like) from about 10 to about 100 nm (e.g., about 10 to about 90, about 10 to about 80, about 10 to about 70, about 10 to about 60, about 10 to about 50, about 10 to about 40, about 10 to about 30, about 10 to about 20, about 20 to about 100, about 20 to about 90, about 20 to about 80, about 20 to about 70, about 20 to about 60, about 20 to about 50, about 20 to about 40, about 20 to about 30, about 30 to about 100, about 30 to about 90, about 30 to about 80, about 30 to about 70, about 30 to about 60, about 30 to about 50, about 30 to about 40, about 40 to about 100, about 40 to about 90, about 40 to about 80, about 40 to about 70, about 40 to about 60, about 40 to about 50, about 50 to about 100, about 50 to about 90, about 50 to about 80, about 50 to about 70, about 50 to about 60, about 60 to about 100, about 60 to about 90, about 60 to about 80, about 60 to about 70, about 70 to about 100, about 70 to about 90, about 70 to about 80, about 80 to about 100, about 80 to about 90, or about 90 to about 100 nm). In some embodiments, the silk nanofiber gelator comprises silk or silk fibroin.

In some embodiments, the peptide nanofiber gelator comprises a peptide sequence derived from silk fibroin or a fragment thereof. In some embodiments, the peptide sequence comprises GAGAGAGY (SEQ ID NO: 4), GAGAGY (SEQ ID NO: 5), GAGAGV (SEQ ID NO: 6), or GAGAGVGY (SEQ ID NO: 7). In some embodiments, the peptide sequence further comprises a hydrocarbon moiety, a fatty acid moiety, an ester of a fatty acid moiety, or an aromatic moiety. In some embodiments, the hydrocarbon moiety comprises lauryl (C12), capryl (C10), or caprylyl (C8); wherein the fatty acid moiety comprises lauric acid, caprylic acid, or capric acid; or wherein the ester of the fatty acid moiety comprises laurate, caprylate, or caprate. In some embodiments, the aromatic moiety comprises naphthyl (Nap), fluorenylmethoxycarbonyl (Fmoc), fluorenyl, anthryl, phenanthryl, indenyl, or pyrenyl (Py).

In some embodiments, the providing step can include: (i) contacting the biological sample with a pre-gel solution that is disposed on a surface of an initial substrate comprising the plurality of capture probes, wherein the pre-gel solution comprises a plurality of gelators; and (ii) applying an external field to the pre-gel solution, thereby providing the hydrogel comprising the plurality of anisotropic structures and providing the biological sample on the substrate. In some embodiments, the providing step can include: (i) contacting the biological sample with a pre-gel solution that is disposed on a surface of an initial substrate comprising the plurality of capture probes, wherein the pre-gel solution comprises a plurality of gelators; (ii) applying an external field to the pre-gel solution, thereby providing an anisotropic phase within the pre-gel solution; and (iii) applying a reagent to promote gelation of the pre-gel solution, thereby providing the hydrogel comprising the plurality of anisotropic structures and providing the biological sample on the substrate. In some embodiments, the reagent comprises a salt (e.g., a divalent salt, such as a Ca²⁺ or Mg²⁺ salt) or an acidifier (e.g., an acid or glucono-delta-lactone). Any of these providing steps can further include concentrating the pre-gel solution, diluting the pre-gel solution, dialyzing of the pre-gel solution, annealing of the pre-gel solution or the hydrogel (e.g., at an elevated temperature from about 30° C. to 65° C.), and/or desiccating the pre-gel solution or the hydrogel.

Gelation or polymerization of the hydrogel can be promoted in any useful manner. In some embodiments, gelation can include the use of a reagent to promote gelation of the pre-gel solution. For example and without limitation, the reagent can include a cation, an anion, or a crosslinking agent that forms bonds (e.g., covalent or noncovalent bonds) between polymeric subunits within the polymeric network. In another non-limiting example, gelation can include concentration and/or dilution of a pre-gel solution to promote the formation of a polymeric network. In yet another non-limiting example, gelation can include application of an external field (e.g., any described herein).

In some embodiments, the plurality of anisotropic structures in the hydrogel comprises a polymeric network formed by assembling a plurality of gelators, thereby forming an anisotropic phase. In some embodiments, the assembling comprises polymerizing the plurality of gelators. In some embodiments, the assembling comprises aligning the plurality of gelators and then polymerizing the plurality of gelators. In some embodiments, the polymerizing and/or the aligning comprises applying an external field. In some embodiments, the external field comprises a magnetic field, an electric field, or an electromagnetic field. In some embodiments, the external field comprises a magnetic field with an example strength of 10 Tesla (T), wherein the magnetic field is sufficiently parallel to the substrate. In some embodiments, the external field comprises a direct current (DC) electric field with an example strength of 50 volts (V), wherein the DC electric field is sufficiently parallel to the substrate. In some embodiments, the external field comprises an electromagnetic field that is sufficiently parallel to the substrate.

Such external fields can include constant or oscillating fields. In some embodiments, the external field is applied in a direction that is sufficiently perpendicular to a surface of the substrate or a surface of the second substrate, if present. In some embodiments, the external field is applied in a direction that is sufficiently parallel to a surface of the substrate or a surface of the second substrate, if present. In some embodiments, the anisotropic phase comprises a plurality of gelators aligned in a direction that is sufficiently perpendicular to a surface of the substrate or to a surface of the second substrate, if present. In some embodiments, the substrate, the first substrate, or the second substrate, if present, comprises a non-porous substrate comprising one of glass, silicon, poly-lysine coated material, nitrocellulose, polystyrene, cyclic olefin copolymers (COCs), cyclic olefin polymers (COPs), polypropylene, polyethylene, or polycarbonate.

### Permeabilization of the Biological Sample

In some embodiments, the methods described herein can include a permeabilizing step that comprises contacting the biological sample (e.g., tissue section) with a permeabilization agent. The analytes or proxies thereof then diffuse into or through the anisotropic structure within the hydrogel after exposure to the permeabilization agent. In such cases, the anisotropic structure can facilitate the diffusion of the analytes in the biological sample into the pores of the hydrogel. In some embodiments, the target analytes are able to diffuse through the permeabilizing agent soaked hydrogel and hybridize or bind the capture probes on the other side of the hydrogel.

In some embodiments, the biological sample can be permeabilized to facilitate transfer of analytes out of the sample, and/or to facilitate transfer of species (such as capture probes) into the sample. If a sample is not permeabilized sufficiently, the amount of analyte captured from the sample may be too low to enable adequate analysis. Conversely, if the tissue sample is over permeabilized, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, sodium dodecyl sulfate (SDS), or any surfactant described herein), and enzymes (e.g., trypsin, proteases (e.g., proteinase K). In some embodiments, the detergent is an anionic detergent (e.g., SDS or N-lauroylsarcosine sodium salt solution). In some embodiments, the biological sample can be permeabilized using any of the methods described herein (e.g., using any of the detergents described herein, e.g., SDS and/or N-lauroylsarcosine sodium salt solution) before or after enzymatic treatment (e.g., treatment with any of the enzymes described herein, e.g., trypsin, proteases (e.g., pepsin and/or proteinase K)). In some embodiments, the permeabilization agent comprises proteinase K. In some embodiments, the permeabilization agent is provided in the presence of a buffer (e.g., Tris, as well as any described herein), a surfactant (e.g., an ionic surfactant, such as any described herein), a PEG (e.g., any described herein), or a combination thereof.

In some embodiments, a biological sample can be permeabilized by exposing the sample to greater than about 1.0 w/v % (e.g., greater than about 2.0 w/v %, greater than about 3.0 w/v %, greater than about 4.0 w/v %, greater than about 5.0 w/v %, greater than about 6.0 w/v %, greater than about 7.0 w/v %, greater than about 8.0 w/v %, greater than about 9.0 w/v %, greater than about 10.0 w/v %, greater than about 11.0 w/v %, greater than about 12.0 w/v %, or greater than about 13.0 w/v %) sodium dodecyl sulfate (SDS) and/or N-lauroylsarcosine or N-lauroylsarcosine sodium salt. In some embodiments, a biological sample can be permeabilized by exposing the sample (e.g., for about 5 minutes to about 1 hour, about 5 minutes to about 40 minutes, about 5 minutes to about 30 minutes, about 5 minutes to about 20 minutes, or about 5 minutes to about 10 minutes) to about 1.0 w/v % to about 14.0 w/v % (e.g., about 2.0 w/v % to about 14.0 w/v %, about 2.0 w/v % to about 12.0 w/v %, about 2.0 w/v % to about 10.0 w/v %, about 4.0 w/v % to about 14.0 w/v %, about 4.0 w/v % to about 12.0 w/v %, about 4.0 w/v % to about 10.0 w/v %, about 6.0 w/v % to about 14.0 w/v %, about 6.0 w/v % to about 12.0 w/v %, about 6.0 w/v % to about 10.0 w/v %, about 8.0 w/v % to about 14.0 w/v %, about 8.0 w/v % to about 12.0 w/v %, about 8.0 w/v % to about 10.0 w/v %, about 10.0% w/v % to about 14.0 w/v %, about 10.0 w/v % to about 12.0 w/v %, or about 12.0 w/v % to about 14.0 w/v %) SDS and/or N-lauroylsarcosine salt solution and/or proteinase K (e.g., at a temperature of about 4% to about 35° C., about 4° C. to about 25° C., about 4° C. to about 20° C., about 4° C. to about 10° C., about 10° C. to about 25° C., about 10° C. to about 20° C., about 10° C. to about 15° C., about 35° C. to about 50° C., about 35° C. to about 45° C., about 35° C. to about 40° C., about 40° C. to about 50° C., about 40° C. to about 45° C., or about 45° C. to about 50° C.).

In some embodiments, the biological sample can be incubated with a permeabilizing agent to facilitate permeabilization of the sample. Additional methods for sample permeabilization are described, for example, in Jamur et al., Method Mol. Biol. 588:63-66, 2010, the entire contents of which are incorporated herein by reference.

In some embodiments, the permeabilizing step of the methods described herein can further comprise removing the permeabilized biological sample from the substrate. In some embodiments, the removal of the biological sample can optionally be performed to remove all or a portion of the biological sample from the substrate. In some embodiments, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some embodiments, the removal step can include ablation of the tissue (e.g., laser ablation).

In some embodiments, the permeabilized biological sample is removed from the substrate prior to the analyte, or the proxy thereof, hybridizing to the capture probe. In some embodiments, the permeabilized biological sample is removed from the substrate after the analyte, or the proxy thereof, hybridizes to the capture probe.

In some embodiments, the biological sample (e.g., tissue sample or tissue section) is a fresh and/or frozen biological sample (e.g., tissue sample or tissue section). In some embodiments, the biological sample (e.g., tissue sample or tissue section) is a fixed biological sample (e.g., tissue sample or tissue section). In some embodiments, the fixed biological sample is a formalin-fixed paraffin-embedded biological sample, a PFA fixed biological sample, or an acetone fixed biological sample.

In some embodiments, the methods described herein can further comprise staining and/or imaging the biological sample. In some embodiments, the methods described herein can further comprise second strand synthesis. In some embodiments, the methods described herein can further comprise sequencing. In some embodiments, the determining step of the methods described herein, if present, can comprise sequencing. In some embodiments, the sequencing comprises sequencing a sequence of the spatial barcode or a complement thereof; all or a portion of a sequence of the analyte from the biological sample, or a complement thereof; all or a part of a sequence of the nucleic acid of the biological sample bound to the capture domain, or a complement thereof.

### D. Kits

In some embodiments, also provided herein are spatial arrays that include: (a) a substrate; (b) a hydrogel comprising a plurality of anisotropic structures, wherein the plurality of anisotropic structures in the hydrogel comprises a polymeric network formed by assembling a plurality of gelators, thereby forming an anisotropic phase; and (c) a plurality of capture probes disposed between a surface of the substrate and a surface of the hydrogel, wherein at least one capture probe of the plurality of capture probes comprises: (i) a spatial barcode and (ii) a capture moiety.

In some embodiments, also provided herein are kits that include: (a) a plurality of capture probes disposed on a surface of a substrate, wherein at least one capture probe of the plurality of capture probes comprises: (i) a spatial barcode and (ii) a capture moiety; and (b) a hydrogel or a pre-gel solution, wherein the hydrogel comprises a plurality of anisotropic structures in a polymeric network formed by assembling a plurality of gelators, or wherein the pre-gel solution comprises a plurality of gelators. In some embodiments, a kit can further include: (c) instructions for performing any one of the methods described herein.

In some embodiments, the substrate can comprise any one of the spatial arrays described herein. In some embodiments, the substrate is configured to provide a biological sample. In some embodiments, the biological sample is a fixed biological sample or a fresh frozen biological sample. In some embodiments, the fixed biological sample is a formalin-fixed paraffin-embedded biological sample, a PFA fixed biological sample, or an acetone fixed biological sample.

In some embodiments, the kit can further include instructions for performing staining and/or imaging of the biological sample. In some embodiments, the kit can further include one or more permeabilization reagents, reverse transcription (RT) reagents, second strand reagents, or amplification reagents. In some embodiments, the kit can further include instructions for performing second strand synthesis or amplification.

### EXAMPLES

### Example 1—Transcript Mislocalization (TML) Mitigation Based on Anisotropic Structures

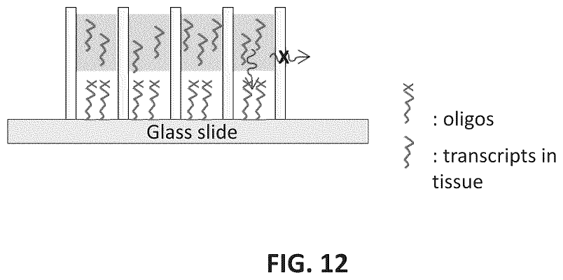

TML can be a cause of lateral diffusion of transcripts in spatial analysis. Described herein are methods of TML mitigation by introducing anisotropic structures on a substrate. FIG. 12 depicts an exemplary arrangement of anisotropic structures to reduce the lateral diffusion of transcripts from the tissue without lowering the diffusion rate of transcripts towards the capture probes disposed on a surface of the substrate (here, a non-limiting glass slide). Reducing lateral diffusion may, in some instances, enhance sensitivity and resolution of spatial analysis.

In some embodiments, the anisotropic structures can be hydrogels with anisotropic structures. Furthermore, such anisotropic structures can be incorporated either by pre-printing the hydrogel on a substrate or by providing instructions to a user to add a pre-gel solution to a substrate to generate the hydrogel.

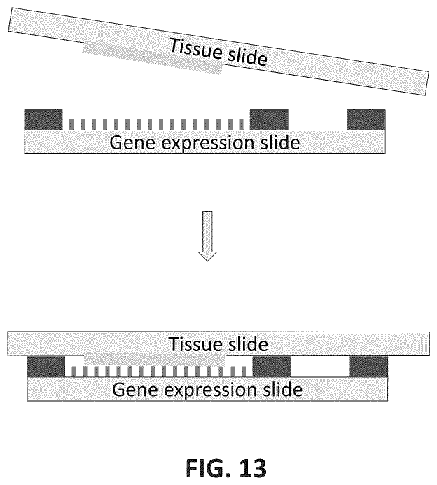

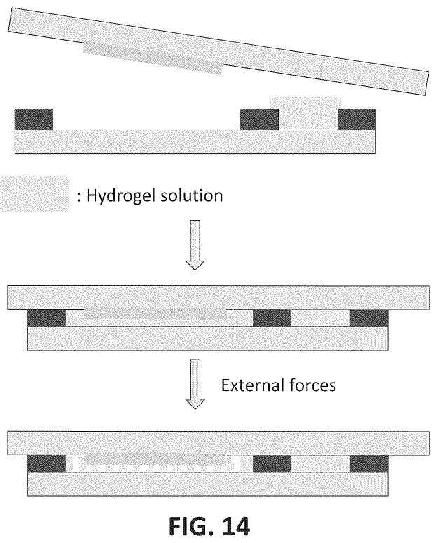

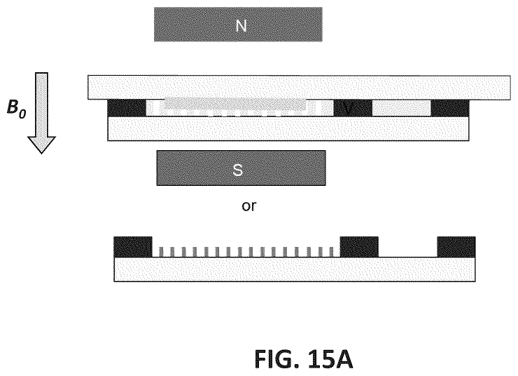

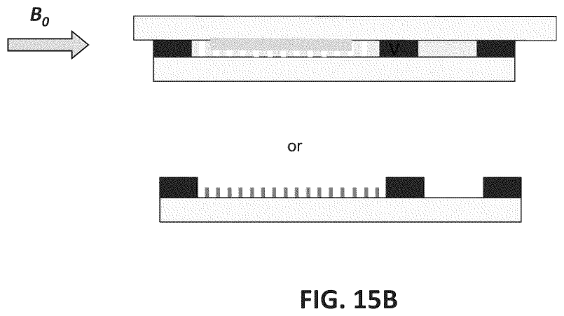

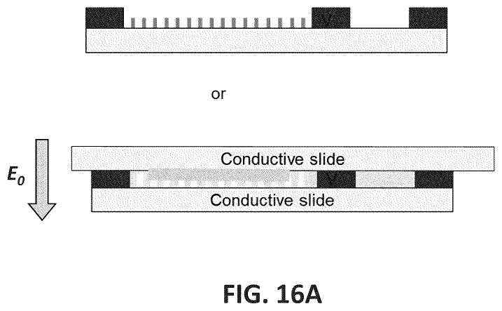

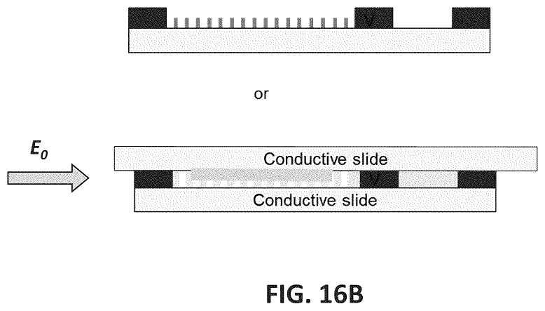

Anisotropic structures can be implemented with any sandwiching process herein. FIG. 13 shows an exemplary sandwiching process where a first substrate (e.g., a tissue slide), including a biological sample, and a second substrate (e.g., a spatial array having spatially barcoded capture probes) are brought into proximity with one another. A hydrogel can be introduced on the second substrate prior to aligning the first substrate and second substrate. FIG. 14 shows an exemplary sandwiching process in which a pre-gel solution is introduced, the first and second substrates are aligned, and then an external field is applied between, across, or to the first and second substrates. FIG. 15A and FIG. 15B show exemplary sandwich configurations in which a magnetic field (B₀) is applied. Such a field can be applied in any direction. FIG. 16A and FIG. 16B show exemplary sandwich configurations in which an electric field (E₀) is applied. Such a field can be applied in any direction. In some embodiments, the first and second substrates are coated with or formed of a material that accommodates or sustains an electric field (e.g., a conductive substrate or a substrate having a conductive coating (e.g., Indium Tin Oxide; ITO)). The methods herein can include those that provide a hydrogel comprising a plurality of anisotropic structures disposed between the biological sample and a surface of the substrate comprising the plurality of capture probes, as well as methods of using a substrate having such a hydrogel.

### Embodiments

Embodiment (E)1. A method for determining the location of an analyte in a biological sample, the method comprising:
(a) providing the biological sample on a substrate, wherein the substrate comprises a plurality of capture probes, and wherein a hydrogel comprising a plurality of anisotropic structures is disposed between the biological sample and a surface of the substrate comprising the plurality of capture probes;
(b) hybridizing or contacting the analyte, or a proxy thereof, with at least one capture probe of the plurality of capture probes, wherein the at least one capture probe comprises a capture domain and a spatial barcode; and
(c) determining (i) the spatial barcode or a complement thereof, and (ii) all or a portion of the analyte from the biological sample, and using the determined sequences of (i) and (ii) to determine the location of the analyte in the biological sample.

E2. The method of E1, wherein the providing step (a) comprises:
(i) providing an initial substrate comprising the plurality of capture probes and the hydrogel comprising the plurality of anisotropic structures; and
(ii) contacting the biological sample with the hydrogel, thereby providing the biological sample on the substrate.

E3. The method of E2, wherein the hydrogel comprising the plurality of anisotropic structures is prepared by (i) applying an external field to a pre-gel solution comprising a plurality of gelators, optionally wherein the pre-gel solution is disposed on a surface of the initial substrate, and (ii) optionally applying a reagent to promote gelation of the pre-gel solution, thereby providing the hydrogel comprising the plurality of anisotropic structures.

E4. The method of E1, wherein the providing step (a) comprises:
(i) contacting the biological sample with a pre-gel solution that is disposed on a surface of an initial substrate comprising the plurality of capture probes, wherein the pre-gel solution comprises a plurality of gelators;
(ii) applying an external field to the pre-gel solution, thereby providing the hydrogel comprising the plurality of anisotropic structures and providing the biological sample on the substrate.

E5. The method of E1, wherein the providing step (a) comprises:
(i) contacting the biological sample with a pre-gel solution that is disposed on a surface of an initial substrate comprising the plurality of capture probes, wherein the pre-gel solution comprises a plurality of gelators;
(ii) applying an external field to the pre-gel solution, thereby providing an anisotropic phase within the pre-gel solution; and
(iii) applying a reagent to promote gelation of the pre-gel solution, thereby providing the hydrogel comprising the plurality of anisotropic structures and providing the biological sample on the substrate.

E6. The method of E5, wherein the reagent comprises a salt or an acidifier, optionally wherein the salt is selected from a Ca2+ or Mg2+ salt and/or optionally wherein the acidifier is selected from an acid or glucono-delta-lactone.

E7. The method of any one of E1-E6, wherein the biological sample is permeabilized to allow the analyte, or the proxy thereof, in the biological sample to interact with the at least one capture probe of the plurality of capture probes; or wherein the method further comprises permeabilizing the biological sample to allow the analyte, or the proxy thereof, in the biological sample to interact with the at least one capture probe of the plurality of capture probes, wherein the permeabilizing is performed before or during step (b).

E8. A method for determining the location of an analyte in a biological sample, the method comprising:
(a) providing the biological sample on a first substrate;
(b) aligning the first substrate with a second substrate comprising an array and a hydrogel, such that at least a portion of the biological sample is aligned with at least a portion of the array, and such that at least a portion of the hydrogel is disposed between the biological sample and the second substrate, wherein the array comprises a plurality of capture probes, and wherein the hydrogel comprises a plurality of anisotropic structures;
(c) when the biological sample is aligned with at least a portion of the array and at least a portion of the hydrogel, hybridizing or contacting the analyte, or a proxy thereof, with at least one capture probe of the plurality of capture probes, wherein the at least one capture probe comprises a capture domain and a spatial barcode; and
(d) determining (i) the spatial barcode or a complement thereof, and (ii) all or a portion of the analyte from the biological sample, and using the determined sequences of (i) and (ii) to determine the location of the analyte in the biological sample.

E9. The method of E8, further comprising permeabilizing the biological sample to allow the analyte, or the proxy thereof, in the biological sample to interact with at least one capture probe of the plurality of capture probes, wherein the permeabilizing is performed before or during step (c).

E10. A method for determining the location of an analyte in a biological sample, the method comprising:
(a) providing the biological sample on a first substrate;
(b) aligning the first substrate with a second substrate comprising an array, such that at least a portion of the biological sample is aligned with at least a portion of the array, and such that at least a portion of a pre-gel solution is disposed between the biological sample and the second substrate, wherein the array comprises a plurality of capture probes;
(c) applying an external field to the pre-gel solution and optionally applying a reagent to promote gelation of the pre-gel solution, thereby providing a hydrogel comprising a plurality of anisotropic structures;
(d) when the biological sample is aligned with at least a portion of the array and at least a portion of the hydrogel, hybridizing or contacting the analyte, or a proxy thereof, with at least one capture probe of the plurality of capture probes, wherein the at least one capture probe comprises a capture domain and a spatial barcode; and
(e) determining (i) the spatial barcode or a complement thereof, and (ii) all or a portion of the analyte from the biological sample, and using the determined sequences of (i) and (ii) to determine the location of the analyte in the biological sample.

E11. The method of E10, further comprising permeabilizing the biological sample to allow the analyte, or the proxy thereof, in the biological sample to interact with at least one capture probe of the plurality of capture probes, wherein the permeabilizing is performed before or during step (d).

E12. A method for determining the location of a nucleic acid in a biological sample, the method comprising:
(a) contacting the biological sample with a substrate, wherein the substrate comprises:
   (i) a plurality of capture probes and
   (ii) a hydrogel comprising plurality of anisotropic structures, such that the hydrogel is disposed between the biological sample and a surface of the substrate comprising the plurality of capture probes;
(b) hybridizing or contacting the nucleic acid of the biological sample, or a proxy thereof, with at least one capture probe of the plurality of capture probes, wherein the at least one capture probe comprises a capture domain and a spatial barcode;
(c) extending a 3′ end of the at least one capture probe using the nucleic acid of the biological sample, or the proxy thereof, bound to the capture domain as a template to generate an extended capture probe; and
(d) determining (i) all or a part of a nucleotide sequence of the nucleic acid of the biological sample bound to the capture domain, or a complement thereof, and (ii) a nucleotide sequence of the spatial barcode, or a complement thereof, and using the determined nucleotide sequences of (i) and (ii) to determine the location of the nucleic acid in the biological sample.

E13. The method of E12, further comprising permeabilizing the biological sample to allow the nucleic acid in the biological sample, or the proxy thereof, to interact with the at least one capture probe of the plurality of capture probes, wherein the permeabilizing is performed before or during step (b).

E14. The method of E12 or E13, further comprising generating a complement of the extended capture probe.

E15. The method of E14, further comprising releasing the complement of the extended capture probe and optionally amplifying the complement of the extended capture probe.

E16. A method for determining the location of a nucleic acid in a biological sample, the method comprising:
(a) contacting the biological sample with a substrate, wherein the substrate comprises a plurality of capture probes;
(b) contacting the biological sample with a pre-gel solution that is disposed on a surface of the substrate;
(c) applying an external field to the pre-gel solution and optionally applying a reagent to promote gelation of the pre-gel solution, thereby providing a hydrogel comprising a plurality of anisotropic structures;
(d) hybridizing or contacting the nucleic acid of the biological sample, or a proxy thereof, with at least one capture probe of the plurality of capture probes, wherein the at least one capture probe comprises a capture domain and a spatial barcode;
(e) extending a 3′ end of the at least one capture probe using the nucleic acid of the biological sample, or the proxy thereof, bound to the capture domain as a template to generate an extended capture probe; and
(f) determining (i) all or a part of a nucleotide sequence of the nucleic acid of the biological sample bound to the capture domain, or a complement thereof, and (ii) a nucleotide sequence of the spatial barcode, or a complement thereof, and using the determined nucleotide sequences of (i) and (ii) to determine the location of the nucleic acid in the biological sample.

E17. The method of E16, further comprising permeabilizing the biological sample to allow the nucleic acid in the biological sample, or the proxy thereof, to interact with the at least one capture probe of the plurality of capture probes, wherein the permeabilizing is performed before or during step (d).

E18. The method of E16 or E17, further comprising generating a complement of the extended capture probe.

E19. The method of E18, further comprising releasing the complement of the extended capture probe and optionally amplifying the complement of the extended capture probe.

E20. The method of any one of E12-E19, wherein the nucleic acid is DNA.

E21. The method of any one of E12-E19, wherein the nucleic acid is RNA.

E22. A method for mitigating mislocalization of analytes captured on a spatial array, the method comprising:
(a) providing the biological sample on a substrate, wherein the substrate comprises a spatial array comprising a plurality of capture probes, and wherein a hydrogel comprising a plurality of anisotropic structures is disposed between the biological sample and the spatial array; and
(b) hybridizing or capturing an analyte, or the proxy thereof, using at least one capture probe of the plurality of capture probes, wherein the at least one capture probe comprises a capture domain and a spatial barcode, thereby mitigating mislocalization of the captured analyte, or the captured proxy thereof, and its subsequent mislocalization on the spatial array.

E23. The method of E22, wherein step (b) comprises migration of the captured analyte, or the captured proxy thereof, wherein the migration comprises vertical migration between the biological sample and the spatial array.

E24. The method of E22, further comprising:
(c) determining (i) the spatial barcode or a complement thereof, and (ii) all or a portion of the sequence of the analyte from the biological sample.

E25. The method of any one of E1-E24, wherein the plurality of anisotropic structures in the hydrogel comprises a polymeric network formed by assembling a plurality of gelators, thereby forming an anisotropic phase.

E26. The method of E25, wherein the assembling comprises polymerizing the plurality of gelators.

E27. The method of E25, wherein the assembling comprises aligning the plurality of gelators and then polymerizing the plurality of gelators.

E28. The method of E26 or E27, wherein the polymerizing and/or the aligning comprises applying an external field.

E29. The method of E28, wherein the external field comprises a magnetic field, an electric field, or an electromagnetic field.

E30. The method of E29, wherein the external field is applied in a direction that is sufficiently perpendicular to a surface of the substrate or a surface of the second substrate, if present.

E31. The method of E29, wherein the external field is applied in a direction that is sufficiently parallel to a surface of the substrate or a surface of the second substrate, if present.

E32. The method of E25, wherein the anisotropic phase comprises a plurality of gelators aligned in a direction that is sufficiently perpendicular to a surface of the substrate or to a surface of the second substrate, if present.

E33. The method of E1-E32, wherein the hydrogel comprises a network formed from a plurality of gelators, optionally wherein the plurality of gelators comprises a hydrogelator or a self-assembled gelator.

E34. The method of E1-E33, wherein the pre-gel solution, if present, comprises a plurality of gelators.

E35. The method of E33 or E34, wherein the gelator comprises a ferromagnetic gelator, a paramagnetic gelator, a low molecular weight gelator optionally with a molecular weight of less than 3000 Daltons, an amphiphilic gelator, a nanofiber gelator, or a polymer-derived gelator.

E36. The method of E35, wherein the low molecular weight gelator comprises a peptide comprising an aromatic moiety, optionally wherein the peptide comprises a dipeptide or a tripeptide.

E37. The method of E36, wherein the aromatic moiety comprises naphthyl (Nap), fluorenylmethoxycarbonyl (Fmoc), fluorenyl, anthryl, phenanthryl, indenyl, or pyrenyl (Py).

E38. The method of E36, wherein the peptide comprises phenylalanine (Phe), lysine (Lys), tyrosine (Tyr), cyclohexylalanine (Cha), or a combination thereof.

E39. The method of E36, wherein the peptide comprises NapPhePhe, NapPhePhePhe, NapPhePhePheLys (Nap-SEQ ID NO: 1), NapPhePheLysLys (Nap-SEQ ID NO: 2), NapPhePhePheLysTyr, FmocPhe (Nap-SEQ ID NO: 3-Fmoc), FmocTyr, FmocPhePhe, or FmocPhePheLysLys (Fmoc-SEQ ID NO: 2).

E40. The method of E35, wherein the nanofiber gelator comprises a silk nanofiber gelator or a peptide nanofiber gelator.

E41. The method of E40, wherein the silk nanofiber gelator comprises a dimension from about 10 to 100 nm, optionally wherein the dimension is a diameter or width.

E42. The method of E40, wherein the silk nanofiber gelator comprises silk or silk fibroin.

E43. The method of E40, wherein the peptide nanofiber gelator comprises a peptide sequence derived from silk fibroin or a fragment thereof.

E44. The method of E43, wherein the peptide sequence comprises GAGAGAGY (SEQ ID NO: 4), GAGAGY (SEQ ID NO: 5), GAGAGV (SEQ ID NO: 6), or GAGAGVGY (SEQ ID NO: 7).

E45. The method of E43 or E44, wherein the peptide sequence further comprises a hydrocarbon moiety, a fatty acid moiety, an ester of a fatty acid moiety, or an aromatic moiety.

E46. The method of E45, wherein the hydrocarbon moiety comprises lauryl (C12), capryl (C10), or caprylyl (C8); wherein the fatty acid moiety comprises lauric acid, caprylic acid, or capric acid; or wherein the ester of the fatty acid moiety comprises laurate, caprylate, or caprate.

E47. The method of E45, wherein the aromatic moiety comprises naphthyl (Nap), fluorenylmethoxycarbonyl (Fmoc), fluorenyl, anthryl, phenanthryl, indenyl, or pyrenyl (Py).

E48. The method of any of E1-E47, wherein the substrate, the first substrate, or the second substrate, if present, comprises a non-porous substrate comprising one of glass, silicon, poly-lysine coated material, nitrocellulose, polystyrene, cyclic olefin copolymers (COCs), cyclic olefin polymers (COPs), polypropylene, polyethylene, or polycarbonate.

E49. The method of any one of E1-E48, wherein the biological sample is a fixed biological sample.

E50. The method of E49, wherein the fixed biological sample is a formalin-fixed paraffin-embedded biological sample, a PFA fixed biological sample, or an acetone fixed biological sample.

E51. The method of any one of E1-E50, further comprising, staining and/or imaging the biological sample.

E52. The method of any one of E1-E51, further comprising second strand synthesis.

E53. The method of any one of E1-E52, further comprising sequencing.

E54. The method of any one of E1-E53, wherein the determining step comprises sequencing.

E55. The method of E53 or E54, wherein the sequencing comprises sequencing a sequence of the spatial barcode or a complement thereof; all or a portion of a sequence of the analyte from the biological sample, or a complement thereof; all or a part of a sequence of the nucleic acid of the biological sample bound to the capture domain, or a complement thereof.

E56. A spatial array comprising:
(a) a substrate;
(b) a hydrogel comprising a plurality of anisotropic structures, wherein the plurality of anisotropic structures in the hydrogel comprises a polymeric network formed by assembling a plurality of gelators, thereby forming an anisotropic phase; and
(c) a plurality of capture probes disposed between a surface of the substrate and a surface of the hydrogel, wherein at least one capture probe of the plurality of capture probes comprises: (i) a spatial barcode and (ii) a capture moiety.

E57. A kit comprising:
(a) a plurality of capture probes disposed between a surface of a substrate, wherein at least one capture probe of the plurality of capture probes comprises: (i) a spatial barcode and (ii) a capture moiety; and
(b) a hydrogel or a pre-gel solution, wherein the hydrogel comprises a plurality of anisotropic structures in a polymeric network formed by assembling a plurality of gelators, or wherein the pre-gel solution comprises a plurality of gelators.

E58. The kit of E57, further comprising:
(c) instructions for performing a method of any one of E1-E55.

E59. The kit of E57 or E58, wherein the substrate comprises a spatial array of E56.

E60. The kit of any one of E57-E59, wherein the substrate is configured to provide a biological sample.

E61. The kit of E60, wherein the biological sample is a fixed biological sample.

E62. The kit of E61, wherein the fixed biological sample is a formalin-fixed paraffin-embedded biological sample, a PFA fixed biological sample, or an acetone fixed biological sample.

E63. The kit of any one of E57-E62, further comprising instructions for performing staining and/or imaging of the biological sample.

E64. The kit of any one of E57-E63, further comprising one or more permeabilization reagents, reverse transcription (RT) reagents, second strand reagents, or amplification reagents.

E65. The kit of any one of E57-E64, further comprising instructions for performing second strand synthesis or amplification.

What is claimed is:

1. A method for determining the location of an analyte in a biological sample, the method comprising:
(a) providing the biological sample on a substrate, wherein the substrate comprises a plurality of capture probes, and wherein a hydrogel comprising a plurality of anisotropic structures is disposed between the biological sample and a surface of the substrate comprising the plurality of capture probes;
(b) hybridizing or contacting the analyte, or a proxy thereof, with at least one capture probe of the plurality of capture probes, wherein the at least one capture probe comprises a capture domain and a spatial barcode; and
(c) determining (i) the spatial barcode or a complement thereof, and (ii) all or a portion of the analyte from the biological sample, and using the determined sequences of (i) and (ii) to determine the location of the analyte in the biological sample.

2. The method of claim 1, wherein the providing step (a) comprises:
(i) providing an initial substrate comprising the plurality of capture probes and the hydrogel comprising the plurality of anisotropic structures; and
(ii) contacting the biological sample with the hydrogel, thereby providing the biological sample on the substrate.

3. The method of claim 2, wherein the hydrogel comprising the plurality of anisotropic structures is prepared by (i) applying an external field to a pre-gel solution comprising a plurality of gelators, optionally wherein the pre-gel solution is disposed on a surface of the initial substrate, and (ii) optionally applying a reagent to promote gelation of the pre-gel solution, thereby providing the hydrogel comprising the plurality of anisotropic structures.

4. The method of claim 1, wherein the providing in step (a) comprises:
(i) contacting the biological sample with a pre-gel solution that is disposed on a surface of an initial substrate comprising the plurality of capture probes, wherein the pre-gel solution comprises a plurality of gelators;
(ii) applying an external field to the pre-gel solution, thereby providing the hydrogel comprising the plurality of anisotropic structures and providing the biological sample on the substrate.

5. The method of claim 1, wherein the providing step (a) comprises:
(i) contacting the biological sample with a pre-gel solution that is disposed on a surface of an initial substrate comprising the plurality of capture probes, wherein the pre-gel solution comprises a plurality of gelators;
(ii) applying an external field to the pre-gel solution, thereby providing an anisotropic phase within the pre-gel solution; and
(iii) applying a reagent to promote gelation of the pre-gel solution, thereby providing the hydrogel comprising the plurality of anisotropic structures and providing the biological sample on the substrate.

6. The method of claim 1, wherein the plurality of anisotropic structures in the hydrogel comprises a polymeric network formed by assembling a plurality of gelators, thereby forming an anisotropic phase.

7. The method of claim 6, wherein the assembling comprises polymerizing the plurality of gelators.

8. The method of claim 7, further comprising aligning the plurality of gelators and then polymerizing the plurality of gelators.

9. The method of claim 8, wherein the polymerizing and/or the aligning comprises applying an external field.

10. The method of claim 9, wherein the external field comprises a magnetic field, an electric field, or an electromagnetic field.

11. The method of claim 10, wherein the external field is applied in a direction that is sufficiently perpendicular to a surface of the substrate.

12. The method of claim 10, wherein the external field is applied in a direction that is sufficiently parallel to a surface of the substrate.

13. The method of claim 6, wherein the anisotropic phase comprises a plurality of gelators aligned in a direction that is sufficiently perpendicular to a surface of the substrate.

14. The method of claim 1, wherein the hydrogel comprises a network formed from a plurality of gelators, optionally wherein the plurality of gelators comprises a hydrogelator or a self-assembled gelator.

15. The method of claim 3, wherein the pre-gel solution comprises a plurality of gelators.

16. The method of claim 15, wherein the plurality of gelators comprises a ferromagnetic gelator, a paramagnetic gelator, a low molecular weight gelator optionally with a molecular weight of less than 3000 Daltons, an amphiphilic gelator, a nanofiber gelator, or a polymer-derived gelator.

17. The method of claim 16, wherein the low molecular weight gelator comprises a peptide comprising an aromatic moiety, optionally wherein the peptide comprises a dipeptide or a tripeptide.

18. A kit comprising:
(a) a plurality of capture probes disposed on a surface of a substrate, wherein at least one capture probe of the plurality of capture probes comprises: (i) a spatial barcode and (ii) a capture moiety;
(b) a hydrogel or a pre-gel solution, wherein the hydrogel comprises a plurality of anisotropic structures in a polymeric network formed by assembling a plurality of gelators, or wherein the pre-gel solution comprises a plurality of gelators; and
(c) instructions for performing the method of claim 1.

19. A method for determining the location of an analyte in a biological sample, the method comprising:
(a) providing the biological sample on a first substrate;
(b) aligning the first substrate with a second substrate comprising an array and a hydrogel, such that at least a portion of the biological sample is aligned with at least a portion of the array, and such that at least a portion of the hydrogel is disposed between the biological sample and the second substrate, wherein the array comprises a plurality of capture probes, and wherein the hydrogel comprises a plurality of anisotropic structures;
(c) when the biological sample is aligned with at least a portion of the array and at least a portion of the hydrogel, hybridizing or contacting the analyte, or a proxy thereof, with at least one capture probe of the plurality of capture probes, wherein the at least one capture probe comprises a capture domain and a spatial barcode; and
(d) determining (i) the spatial barcode or a complement thereof, and (ii) all or a portion of the analyte from the biological sample, and using the determined sequences of (i) and (ii) to determine the location of the analyte in the biological sample.

20. A spatial array comprising:
(a) a substrate;
(b) a hydrogel comprising a plurality of anisotropic structures, wherein the plurality of anisotropic structures in the hydrogel comprises a polymeric network formed by assembling a plurality of gelators, thereby forming an anisotropic phase; and
(c) a plurality of capture probes disposed between a surface of the substrate and a surface of the hydrogel, wherein at least one capture probe of the plurality of capture probes comprises: (i) a spatial barcode and (ii) a capture domain.


SEQUENCE LISTING (7 sequences)

SEQ ID NO: 1 - protein, 4 aa, synthetic construct
FFFK

SEQ ID NO: 2 - protein, 4 aa, synthetic construct
FFKK

SEQ ID NO: 3 - protein, 6 aa, synthetic construct
FFFKYF

SEQ ID NO: 4 - protein, 8 aa, synthetic construct
GAGAGAGY

SEQ ID NO: 5 - protein, 6 aa, synthetic construct
GAGAGY

SEQ ID NO: 6 - protein, 6 aa, synthetic construct
GAGAGV

SEQ ID NO: 7 - protein, 8 aa, synthetic construct
GAGAGVGY